(19) United States
(12) Patent Application Publication
(10) Pub. No.: US 2018/0085319 A1
(43) Pub. Date: Mar. 29, 2018

(54) METHODS AND COMPOSITIONS FOR TREATING CANCER

(71) Applicant: Takashi Kei Kishimoto, Lexington, MA (US)

(72) Inventor: Takashi Kei Kishimoto, Lexington, MA (US)

(21) Appl. No.: 15/717,710

(22) Filed: Sep. 27, 2017

Related U.S. Application Data
(60) Provisional application No. 62/412,786, filed on Oct. 25, 2016.
(60) Provisional application No. 62/410,226, filed on Oct. 19, 2016.
(60) Provisional application No. 62/405,221, filed on Oct. 6, 2016.
(60) Provisional application No. 62/404,754, filed on Oct. 5, 2016.
(60) Provisional application No. 62/403,889, filed on Oct. 4, 2016.
(60) Provisional application No. 62/400,609, filed on Sep. 27, 2016.

(51) Int. Cl. A61K 9/51; A61K 31/436; C07K 16/30; A61K 47/68

(52) U.S. Cl. CPC A61K 9/5153; A61K 31/436; C07K 2317/21; A61K 47/6851; C07K 2317/55; C07K 16/30

(57) ABSTRACT

Provided herein are methods, and related compositions, for treating cancer. For example, a method for creating a neoplasia-neutral tolerogenic environment in a subject, such as one with cancer, and administering a recombinant immunotoxin is provided.

### RELATED APPLICATIONS

This application claims benefit of priority under 35 U.S.C. § 119 to U.S. Provisional Application No. 62/400,609 filed Sep. 27, 2016, U.S. Provisional Application No. 62/403,889 filed Oct. 4, 2016, U.S. Provisional Application No. 62/404,754 filed Oct. 5, 2016, U.S. Provisional Application No. 62/405,221 filed Oct. 6, 2016, U.S. Provisional Application No. 62/410,226 filed Oct. 19, 2016, and U.S. Provisional Application No. 62/412,786 filed Oct. 25, 2016, the entire contents of each of which are incorporated herein by reference.

### FIELD OF THE INVENTION

Provided herein are methods, and related compositions, for treating cancer. For example, a method for creating a neoplasia-neutral tolerogenic environment in a subject, such as one with cancer, and administering a recombinant immunotoxin is provided.

### SUMMARY OF THE INVENTION

In one aspect, a method for treating a subject with a cancer, comprising creating a neoplasia-neutral tolerogenic environment in the subject, and administering recombinant immunotoxin to the subject to treat the cancer is provided.

In one embodiment of any one of the methods or compositions provided herein, the cancer is a non-hematologic cancer. In one embodiment of any one of the methods or compositions provided herein, the cancer comprises mesothelin-expressing cancer cells. In one embodiment of any one of the methods or compositions provided herein, the cancer is mesothelioma, pancreatic adenocarcinoma, ovarian cancer, lung adenocarcinoma, breast cancer or gastric cancer.

In one embodiment of any one of the methods provided herein, the recombinant immunotoxin when administered to the subject, or a test subject, without any immunosuppressive therapy generates or is expected to generate an unwanted immune response in the subject, or test subject. In one embodiment of any one of the methods provided herein, the recombinant immunotoxin when administered to the subject, or a test subject, without any synthetic nanocarriers comprising an immunosuppressant generates or is expected to generate an unwanted immune response in the subject, or test subject.

In one embodiment of any one of the methods provided herein, the unwanted immune response is unwanted antibody production against the recombinant immunotoxin. In one embodiment of any one of the methods provided herein, the unwanted immune response is unwanted antibody production against the toxin of the recombinant immunotoxin.

In one embodiment of any one of the methods provided herein, the neoplasia-neutral tolerogenic environment in the subject is created by administration of synthetic nanocarriers comprising an immunosuppressant to the subject.

In one embodiment of any one of the methods provided herein, the neoplasia-neutral tolerogenic environment that is created is one in which an unwanted immune response against the recombinant immunotoxin is reduced or eliminated while not enhancing the growth of the cancer.

In one embodiment of any one of the methods provided herein, the administration of the recombinant immunotoxin is repeated. In one embodiment of any one of the methods provided herein, the administration of the recombinant immunotoxin is repeated at least 2, 3 or more times.

In one embodiment of any one of the methods provided herein, the neoplasia-neutral tolerogenic environment is present during each administration of the recombinant immunotoxin. In one embodiment of any one of the methods provided herein, the neoplasia-neutral tolerogenic environment is created during each administration of the recombinant immunotoxin.

In one embodiment of any one of the methods provided herein, synthetic nanocarriers comprising an immunosuppressant are administered at least once to the subject during the repeated administrations of the recombinant immunotoxin. In one embodiment of any one of the methods provided herein, synthetic nanocarriers comprising an immunosuppressant are administered at least twice to the subject during the repeated administrations of the recombinant immunotoxin. In one embodiment of any one of the methods provided herein, synthetic nanocarriers comprising an immunosuppressant are administered at least three times to the subject during the repeated administrations of the recombinant immunotoxin.

In one embodiment of any one of the methods provided herein, the synthetic nanocarriers comprising an immunosuppressant are administered with only the first of the administrations of the recombinant immunotoxin.

In one embodiment of any one of the methods provided herein, wherein, when there are at least two administrations of the recombinant immunotoxin, the synthetic nanocarriers comprising an immunosuppressant are administered with only the first and second of the administrations.

In one embodiment of any one of the methods provided herein, synthetic nanocarriers comprising an immunosuppressant are administered with each administration of the recombinant immunotoxin. In one embodiment of any one of the methods provided herein, the administration(s) of the synthetic nanocarriers comprising an immunosuppressant are concomitant with an administration of the recombinant immunotoxin. In one embodiment of any one of the methods provided herein, the administration(s) of the synthetic nanocarriers comprising an immunosuppressant are simultaneous with an administration of the recombinant immunotoxin. In one embodiment of any one of the methods provided herein, the synthetic nanocarriers are administered prior to the recombinant immunotoxin.

In one embodiment of any one of the methods provided herein, the method further comprises administering the recombinant immunotoxin without the synthetic nanocarriers comprising an immunosuppressant. In one embodiment of any one of the methods provided herein, the recombinant immunotoxin is administered without the synthetic nanocarriers comprising an immunosuppressant at least 2, 3 or more times.

In one embodiment of any one of the methods provided herein, there are at least 2 or 3 cycles of the repeated administrations of the recombinant immunotoxin in combination with the synthetic nanocarriers comprising an immunosuppressant, each cycle of repeated administrations being as defined in any one set of repeated administrations as defined in any one of the methods provided herein.

In one embodiment of any one of the methods provided herein, the method further comprises administering the recombinant immunotoxin without the synthetic nanocarriers comprising an immunosuppressant after the at least 2 or 3 cycles. In one embodiment of any one of the methods provided herein, the recombinant immunotoxin is administered without the synthetic nanocarriers comprising an immunosuppressant at least 2, 3 or more times after the at least 2 or 3 cycles.

In one embodiment of any one of the methods or compositions provided herein, the recombinant immunotoxin comprises an antibody, or antigen-binding fragment thereof, and a toxin. In one embodiment of any one of the methods or compositions provided herein, the ligand, such as an antibody, or antigen-binding fragment thereof, of the recombinant immunotoxin specifically binds an antigen expressed on cells of the cancer. In one embodiment of any one of the methods or compositions provided herein, the antigen is mesothelin.

In one embodiment of any one of the methods or compositions provided herein, the toxin of the recombinant immunotoxin is a toxin of bacterial origin. In one embodiment of any one of the methods or compositions provided herein, the toxin of bacterial origin is a Pseudomonas toxin. In one embodiment of any one of the methods or compositions provided herein, the toxin is Pseudomonas exotoxin A. In one embodiment of any one of the methods or compositions provided herein, the recombinant immunotoxin is LMB-100.

In one embodiment of any one of the methods provided herein, the method further comprises administering a checkpoint inhibitor concomitantly with at least one administration of the recombinant immunotoxin. In one embodiment of any one of the methods provided herein, the checkpoint inhibitor is not administered simultaneously with the at least one administration of the recombinant immunotoxin. In one embodiment of any one of the methods provided herein, the checkpoint inhibitor is administered within 24 hours of the at least one administration of the recombinant immunotoxin. In one embodiment of any one of the methods provided herein, the checkpoint inhibitor is administered concomitantly with each administration of the recombinant immunotoxin. In one embodiment of any one of the methods provided herein, the administration or each administration of the checkpoint inhibitor is administered subsequent to an administration or each administration of the recombinant immunotoxin.

In one embodiment of any one of the methods or compositions provided herein, the checkpoint inhibitor is an anti-CLTA4 antibody. In one embodiment of any one of the methods or compositions provided herein, the checkpoint inhibitor is an anti-OX-40 antibody.

In one embodiment of any one of the methods provided herein, the neoplasia-neutral tolerogenic environment is created after administration of the recombinant immunotoxin without an immunosuppressive therapy. In one embodiment of any one of the methods provided herein, an unwanted immune response against the recombinant immunotoxin is present in the subject after an administration of the recombinant immunotoxin without an immunosuppressive therapy.

In one embodiment of any one of the methods provided herein, the method further comprises administering the recombinant immunotoxin without an immunosuppressive therapy to the subject prior to creating a neoplasia-neutral tolerogenic environment. In one embodiment of any one of the methods provided herein, the unwanted immune response is unwanted antibody production against the recombinant immunotoxin. In one embodiment of any one of the methods provided herein, the unwanted immune response is unwanted antibody production against the toxin of the recombinant immunotoxin.

In one embodiment of any one of the methods provided herein, the method further comprises identifying the subject as having the cancer. In one embodiment of any one of the methods provided herein, the subject is one in need of a neoplasia-neutral tolerogenic environment. In one embodiment of any one of the methods provided herein, the method further comprises identifying the subject as being in need of a neoplasia-neutral tolerogenic environment.

In one embodiment of any one of the methods provided herein, the method further comprises assessing an unwanted immune response against the recombinant immunotoxin in the subject.

In one embodiment of any one of the methods or compositions provided herein, the immunosuppressant is an mTOR inhibitor. In one embodiment of any one of the methods or compositions provided herein, the mTOR inhibitor is rapamycin.

In one embodiment of any one of the methods or compositions provided herein, the immunosuppressant is encapsulated in the synthetic nanocarriers.

In one embodiment of any one of the methods or compositions provided herein, the synthetic nanocarriers comprise polymeric nanocarriers. In one embodiment of any one of the methods or compositions provided herein, the polymeric nanocarriers comprise a polyester or a polyester attached to a polyether. In one embodiment of any one of the methods or compositions provided herein, the polyester comprises a poly(lactic acid), poly(glycolic acid), poly(lactic-co-glycolic acid) or polycaprolactone. In one embodiment of any one of the methods or compositions provided herein, the polymeric nanocarriers comprise a polyester and a polyester attached to a polyether. In one embodiment of any one of the methods or compositions provided herein, the polyether comprises polyethylene glycol or polypropylene glycol.

In one embodiment of any one of the methods or compositions provided herein, the mean of a particle size distribution obtained using dynamic light scattering of a population of the synthetic nanocarriers is a diameter greater than 110 nm. In one embodiment of any one of the methods or compositions provided herein, the diameter is greater than 150 nm. In one embodiment of any one of the methods or compositions provided herein, the diameter is greater than 200 nm. In one embodiment of any one of the methods or compositions provided herein, the diameter is greater than 250 nm. In one embodiment of any one of the methods or compositions provided herein, the diameter is less than 5 μm. In one embodiment of any one of the methods or compositions provided herein, the diameter is less than 4 μm. In one embodiment of any one of the methods or compositions provided herein, the diameter is less than 3 μm. In one embodiment of any one of the methods or compositions provided herein, the diameter is less than 2 μm. In one embodiment of any one of the methods or compositions provided herein, the diameter is less than 1 μm. In one embodiment of any one of the methods or compositions provided herein, the diameter is less than 500 nm. In one embodiment of any one of the methods or compositions provided herein, the diameter is less than 450 nm. In one embodiment of any one of the methods or compositions provided herein, the diameter is less than 400 nm. In one embodiment of any one of the methods or compositions provided herein, the diameter is less than 350 nm. In one embodiment of any one of the methods or compositions provided herein, the diameter is less than 300 nm.

In one embodiment of any one of the methods or compositions provided herein, the load of immunosuppressant comprised in the synthetic nanocarriers, on average across the synthetic nanocarriers, is between 0.1% and 50% (weight/weight). In one embodiment of any one of the methods or compositions provided herein, the load is between 0.1% and 25%. In one embodiment of any one of the methods or compositions provided herein, the load is between 1% and 25%. In one embodiment of any one of the methods or compositions provided herein, the load is between 2% and 25%. In one embodiment of any one of the methods or compositions provided herein, the load is between 2% and 10%.

In one embodiment of any one of the methods or compositions provided herein, an aspect ratio of a population of the synthetic nanocarriers is greater than 1:1, 1:1.2, 1:1.5, 1:2, 1:3, 1:5, 1:7 or 1:10.

In one embodiment of any one of the methods provided herein, the method further comprises assessing in the subject an immune response against the recombinant immunotoxin prior to, during or subsequent to the administering to the subject.

In one embodiment of any one of the methods provided herein, the administering is by intravenous, intraperitoneal, or subcutaneous administration.

In one embodiment of any one of the methods provided herein, the dose of the rIT is less than a dose of the rIT that achieves a similar level of efficacy when not administered concomitantly with synthetic nanocarriers comprising an immunosuppressant as provided herein. In one embodiment of any one of the methods provided herein, the dose of the rIT is at least 10% less. In one embodiment of any one of the methods provided herein, the dose of the rIT is at least 20% less. In one embodiment of any one of the methods provided herein, the method further comprises choosing the dose of the rIT to be less than a dose of the rIT that achieves a similar level of therapeutic efficacy when not administered concomitantly with the synthetic nanocarriers comprising an immunosuppressant.

In one aspect, a kit comprising one or more doses comprising a recombinant immunotoxin and one or more doses comprising synthetic nanocarriers comprising an immunosuppressant is provided.

In one embodiment of any one of the kits provided herein, the kit further comprises one or more doses comprising a checkpoint inhibitor.

In one embodiment of any one of the kits provided herein, the kit further comprises instructions for use. In one embodiment of any one of the kits provided herein, the instructions for use comprise instructions for performing any one of the methods provided herein.

In one embodiment of any one of the kits provided herein, the synthetic nanocarriers comprising an immunosuppressant are as described for any one of such compositions provided herein.

In one embodiment of any one of the kits provided herein, the recombinant immunotoxin is as described for any one of such compositions provided herein.

In another aspect, a composition as described in any one of the methods provided or any one of the Examples is provided. In one embodiment, the composition is any one of the compositions for administration according to any one of the methods provided.

In another aspect, any one of the compositions is for use in any one of the methods provided.

### BRIEF DESCRIPTION OF THE FIGURES

FIG. 1 shows mesothelioma tumor response in patients with the highest overall tumor response in the months following treatment with cyclophosphamide and pentostatin (CP/PS) and SS1P. The top graph shows two treatment cycles with eight patients, the middle graph shows four treatment cycles with one patient, and the bottom graph shows six treatment cycles with one patient.

FIGS. 2A-2F show that a combination of LMB-100 and SVP-R prevents ADA response against LMB-100. FIG. 2A is a ribbon diagram of LMB-100 and an illustration of SVP-R. FIG. 2B shows mice injected 7 times with LMB-100 or a combination of LMB-100 and SVP-R 1, 3, or 7 times (indicated by arrows). Anti-LMB-100 antibodies were evaluated by ELISA (n=8). FIG. 2C shows mice injected with LMB-100 and SVP-R as indicated by arrows (n=7). FIG. 2D shows mice injected with LMB-100 and SVP-R as indicated by arrows. Final mean titer on week 10 is shown (n=7). FIG. 2E shows a neutralization assay using plasma from the mice treated (n=7). KLM-1 cells were seeded and treated with plasma-LMB-100 mixture. Cell viability was assessed after 72 hours. Curves represent mean of 7 viability curves (n=7, six replicas per samples). FIG. 2F shows mice injected with LMB-100 and SVP-R as indicated by arrows (n=8). ELISA plates were coated with LMB-100, Fab or anti-TAC-PE24. Plasma samples from week 6 were evaluated. The dilution factor for 50% of binding is shown. Lines indicate mean error bars SEM. For statistical analysis in FIGS. 2B and 2C, AUC for each curve was calculated and compared using one way ANOVA.

FIGS. 3A-3C show mice weight and AUC after the bi-weekly injections shown in FIG. 2B. FIG. 3A shows female Balb/c mice injected 7 times with LMB-100 (2.5 mg/kg) or a combination of LMB-100 and SVP-R (2.5 mg/kg) 1, 3, or 7 times. Plasma was collected and analyzed for anti-LMB-100 antibodies by ELISA. For statistical analysis, AUC for each curve was calculated and analyzed using one way ANOVA. Error bars SEM, n=8. FIG. 3B shows mice weight before each injection. FIG. 3C shows mice injected with LMB-100 (2.5 mg/kg) and SVP-R (2.5 mg/kg) in biweekly cycles that include three i.v. injections every other day (OOD). Mice weight was evaluated before each injection. Injection time is indicated by the arrows (n=7).

FIG. 4 shows the effect of SVP-R on ADA formation against SS1P parent immunotoxin. Female Balb/c mice were injected with either nine doses of SS1P (0.25 mg/kg), a combination of nine doses of SS1P and three doses of SVP-R (2.5 mg/kg) or vehicle (n=10). Plasma was collected and analyzed for anti-SS1P antibodies by ELISA. For statistical analysis, AUC for each curve was calculated. Error bars show the SEM. Injection time is indicated by the arrows.

FIGS. 5A-5B show the effect of neutralizing antibodies in mice plasma on LMB-100 IC₅₀. Mice were injected with either 15 doses of LMB-100 (2.5 mg/kg), a combination of 15 doses of LMB-100 and six doses of SVP-R (2.5 mg/kg) or vehicle (n=7) per the schedule shown in FIG. 2E. Plasma from the mice was diluted and mixed with LMB-100. KLM-1 cells were seeded in 96-well plates and treated with the plasma-immunotoxin mixture. After 72 hours, cell viability was assessed using WST-8. Viability curves were fitted to each sample, and IC₅₀ was calculated. FIG. 5A shows the IC₅₀ of each sample. FIG. 5B shows the correlation of the titer and the IC₅₀ of each sample. The squares represent plasma samples from LMB-100 treated mice, and the triangles show plasma samples from LMB-100+SVP-R treated mice. Error bars show the SEM. P-value is a comparison of the IC₅₀ using one way ANOVA.

FIGS. 6A-6D show that the combination of LMB-100 with SVP-R induces a specific, transferable, and regulatory T-cell mediated immune response. FIG. 6A shows mice injected three times weekly with LMB-100 (i.v. 2.5 mg/kg) or a combination of LMB-100 with SVP-R (2.5 mg/kg, i.v.). On weeks 4-8, mice were challenged with a weekly dose of LMB-100 (i.v.) and ovalbumin (s.c.). Plasma was collected and analyzed for anti-LMB-100 and anti-OVA antibodies by ELISA. For statistical analysis, AUC for each curve was calculated and analyzed using the Mann-Whitney test. Error bars show the SEM, n=13. FIG. 6B shows mice injected six times with vehicle, LMB-100, SVP-R or both. On week 4, splenocytes from donor mice were isolated and adoptively transferred to recipient naïve mice. Recipient mice were injected with LMB-100 six times. Plasma was collected and analyzed for anti-LMB-100 antibodies by ELISA. Error bars show the SEM, results from two separate experiments with identical schedules were combined (n=5 to 10). FIG. 6C shows mice injected with LMB-100 on days 1, 3, 5, 29, 31, 33, 43, 45 and 47. SVP-R was given on days 1, 3 and 5. Anti-mouse CD-25 depleting antibody (PC61) or isotype control were injected i.p. on days 15 and 16. Titer on day 55 are shown. FIG. 6D shows plasma from mice that were injected seven times with LMB-100 or a combination of LMB-100 and SVP-R. Anti-LMB-100 isotypes were analyzed using sandwich ELISA with subclasses IgG1, IgG2a, IgG2b, IgG3 and IgM specific to LMB-100 (n=8).

FIG. 7 shows the ADA response in donor mice used for adoptive transfer. Mice were injected six times with vehicle, LMB-100 (2.5 mg/kg, i.v.), SVP-R (2.5 mg/kg, i.v.) or a combination of LMB-100 and SVP-R. A plasma sample was taken three days after the last injection.

FIGS. 8A-8D show that LMB-100 and SVP-R co-localize preferentially on dendritic cells and macrophages. FIG. 8A shows the experimental protocol. Dye-conjugated SVP-Cy5 and LMB-100-Alexa488 were injected i.v. alone or in combination (n=3-4 mice per group). Spleen cells were analyzed by FACS 2 hours after injection for dye-conjugate uptake. FIGS. 8B-8C show representative FACS plots show gating for macrophages (F4/80+CD11b+) and dendritic cells (CD11c+MHC-II+), and in vivo uptake by the gated populations. Bold quadrants indicate the percent of positive cells analyzed for each experimental condition. FIG. 8D shows a summary of SVP-R and LMB-100 in vivo uptake by macrophages, DC, monocytes, CD4+ T cells, B cells, neutrophils and CD8+ T cells. The gating strategy for all cells is shown in Table 1.

FIGS. 9A-9C show representative gating strategies of mice splenocytes after injection of LMB-100-Alexa 488 and SVP-R-CY5. Mice were injected consecutively with LMB-100-Alexa488 and SVP-R-Cy5. Two hours post-injection, splenocytes were isolated, labeled and analyzed on a FACS CANTO II flow cytometer. FIG. 9A shows DC and macrophages, FIG. 9B shows B and T cell lymphocytes, and FIG. 9C shows neutrophils and monocytes.

FIGS. 10A-10D show that the combination of LMB-100 with SVP-R induces immune tolerance in mice with pre-existing antibodies specific to the immunotoxin. FIG. 10A shows female BALB/c mice injected six times with LMB-100 (i.v. 2.5 mg/kg) on weeks 1 and 3 to induce a titer of ADA against LMB-100. On week 10, mice were challenged with three doses of either LMB-100, vehicle (PBS) or LMB-100+SVP-R. LMB-100 and SVP-R treated mice were challenged with three additional doses of LMB-100 on week 12. Plasma was collected and analyzed for LMB-100 ADAs by ELISA. Error bars show the SEM, n=7 or 12. FIG. 10B shows female BALB/c mice injected 12 times with LMB-100 over the course of 14 weeks to induce a high titer of ADA against LMB-100. In week 15, mice were immunized with LMB-100 or LMB-100+SVP-R. ADA titers pre- and post-challenge are shown. FIGS. 10C-10D show BM and spleen isolated from mice that had pre-existing ADA and were challenged with either PBS, LMB-100, SVP-R or a combination of LMB-100 and SVP-R. BM cells and splenocytes (100,000 cells/well) were seeded in ELISpot plates that were pre-coated with LMB-100 (n=8).

FIGS. 11A-11B show the development of AB1-L9. FIG. 11A shows a mouse mesothelioma cell line stably transfected with human mesothelin. AB-1 (nonhuman mesothelin transfected, light gray) and AB1-L9 (human mesothelin transfected, dark gray) were labeled with MN antibody, and a secondary PE-labeled antibody. MFI were detected using FACS and analyzed using FLOWJO software. FIG. 11B shows AB1-L9 cells incubated with various concentrations of LMB-100 and evaluated for cell viability using a WST-8 cell counting kit. The experiment was run in three replicas, and the error bars show the SEM.

FIGS. 12A-12F show that the combination of SVP-R with LMB-100 restores neutralized anti-tumor activity. FIG. 12A shows AB1-L9 cells inoculated into mice and treated with PBS, LMB-100, or SVP-R as indicated by arrows (n=7). FIG. 12B shows mice immunized with LMB-100 four times to induce a baseline titer and inoculated with AB1-L9. Mice were treated with vehicle, LMB-100, or LMB-100 and SVP-R as indicated by arrows. Tumor size was measured using a caliper (n=7). FIG. 12C shows plasma from days 5 and 19 analyzed for anti-LMB-100 antibodies by ELISA. Titer was interpolated at 10% of the signal. FIG. 12D shows mice treated as described in FIG. 12C. The experiment was terminated on day 31. The Kaplan-Meyer plot shows time to experimental endpoint (once tumor volume was greater than 400 mm³ or if the mouse lost >30% of its body weight (one mouse)) (n=7). FIG. 12E shows mice inoculated with CT26 cells on day 1 and treated with SVP-R or vehicle on days 10 and 16. Values indicate average tumor size (n=7), error bars show SEM. FIG. 12F shows mice inoculated with 66C14 cells on day 1 and treated with SVP-R or vehicle on days 10, 12 and 14. Values indicate average tumor size (n=5). For statistical analysis, the AUC for each curve was calculated and compared using one way ANOVA. Error bars show SEM.

FIGS. 13A-13B show titer and weight of tumor bearing mice after treatment as shown in FIG. 10A. AB1-L9 cells were inoculated into BALB/c mice. Mice were treated with PBS, LMB-100, SVP-R, or a combination of the latter two on days 7, 9, 11, 14, 16 and 18 (n=7). Error bar shows the SEM. FIG. 13A shows serum samples taken on days 19 and 24 and evaluated for LMB-100 ADA. Due to low general titers, titers were interpolated on 10% of the curve. FIG. 13B shows mice weight throughout the term of immunization.

FIG. 14 shows the weight of tumor bearing mice with pre-existing antibodies to the immunotoxin after treatment as shown in FIG. 10B. BALB/c weight after immunization with LMB-100 four times and inoculation with AB1-L9 is shown. Mice were treated with PBS or LMB-100 on days 5, 7, 9, 12, 14 and 16 and with SVP-R or vehicle on days 5 and 9 (n=7). Error bar shows the SEM.

FIGS. 15A-15D show that SVP-R enhances the cytotoxic activity of LMB-100 in human cell lines. KLM-1 and HAY cells were seeded in 96-well plates and treated with various concentrations of SVP-R, LMB-100 or both. After 72 hours, cell viability was assessed using WST-8 or crystal violet. Viability curves were fitted to each sample, and IC50 was calculated. FIG. 15A shows the cytotoxic activity of SVP-R in both cell lines. FIG. 15B shows the activity of LMB-100 in KLM-1 cells with or without 5 μg/ml of rapamycin encapsulated in SVP. FIG. 15C shows the activity of LMB-100 in HAY cells with or without 1 μg/ml of rapamycin encapsulated in SVP. Curves show a mean of six replicas, error bars show SEM. FIG. 15D shows representative well images taken after HAY cells were fixed and stained with crystal violet.

FIGS. 16A-16B show that SVP-R activity is not diminished by checkpoint inhibitor antibodies. BALB/c mice were immunized weekly with LMB-100 or LMB-100 and SVP-R five times (2.5 mg/kg) (i.v.), and five days after each injection were immunized with anti-mouse CTLA-4 antagonist (FIG. 16A) or anti-OX-40 antagonist (FIG. 16B) or vehicle (i.p.). Plasma samples were collected on day 6 of each week and LMB-100 ADA titer was evaluated using direct ELISA. Error bars show the SEM, n=8. These experiments were repeated with n=5 and n=3, respectively, with similar results.

FIG. 17 shows the inhibition of the anti-LMB-100 antibody responses using LMB-100 and synthetic nanocarriers comprising rapamycin.

FIGS. 18A-18D show anti-LMB-100 antibody titers from serum samples from before and after challenge.

FIG. 19 shows anti-LMB-100 antibody titers for the three groups (bleed 3).

FIG. 20A is a schematic depicting the administration regimen used to examine a syngeneic tumor mouse model (BALB/c mice). FIG. 20B shows tumor sizes of mice undergoing the regimen of FIG. 20A. The first row of arrows (gray) show administration of LMB-100, and the second row of arrows (black) show the administration of rapamycin-comprising nanocarriers.

FIG. 21 shows the weights of mice undergoing the regimen of FIG. 20A. The first row of arrows (gray) show administration of LMB-100, and the second row of arrows (black) show the administration of rapamycin-comprising nanocarriers.

FIG. 22 shows antibody titers and their correlation with tumor size of mice undergoing the regimen of FIG. 20A.

FIG. 23A is a schematic depicting the administration regimen used to examine a syngeneic mesothelin transgenic mouse model. FIG. 23B shows tumor sizes of mice undergoing the regimen of FIG. 23A. The first row of arrows (gray) show administration of LMB-100, and the second row of arrows (black) show the administration of rapamycin-comprising nanocarriers.

FIG. 24 shows the weights of mice undergoing the regimen of FIG. 23A. The first row of arrows (gray) show administration of LMB-100, and the second row of arrows (black) show the administration of rapamycin-comprising nanocarriers.

FIG. 25 shows the antibody titers and their correlation with tumor size of mice undergoing the regimen of FIG. 23A.

FIG. 26 shows peak blood levels of LMB-100 after day 1 LMB-100 infusion during cycle 1 to 4 in subjects with mesothelioma.

### DETAILED DESCRIPTION OF THE INVENTION

Before describing the present invention in detail, it is to be understood that this invention is not limited to particularly exemplified materials or process parameters as such may, of course, vary. It is also to be understood that the terminology used herein is for the purpose of describing particular embodiments of the invention only, and is not intended to be limiting of the use of alternative terminology to describe the present invention.

All publications, patents and patent applications cited herein, whether supra or infra, are hereby incorporated by reference in their entirety for all purposes.

As used in this specification and the appended claims, the singular forms “a,” “an” and “the” include plural referents unless the content clearly dictates otherwise. For example, reference to “a polymer” includes a mixture of two or more such molecules or a mixture of differing molecular weights of a single polymer species, reference to “a synthetic nanocarrier” includes a mixture of two or more such synthetic nanocarriers or a plurality of such synthetic nanocarriers, and the like.

As used herein, the term “comprise” or variations thereof such as “comprises” or “comprising” are to be read to indicate the inclusion of any recited integer (e.g. a feature, element, characteristic, property, method/process step or limitation) or group of integers (e.g. features, elements, characteristics, properties, method/process steps or limitations) but not the exclusion of any other integer or group of integers. Thus, as used herein, the term “comprising” is inclusive and does not exclude additional, unrecited integers or method/process steps.

In embodiments of any one of the compositions and methods provided herein, “comprising” may be replaced with “consisting essentially of” or “consisting of”. The phrase “consisting essentially of” is used herein to require the specified integer(s) or steps as well as those which do not materially affect the character or function of the claimed invention. As used herein, the term “consisting” is used to indicate the presence of the recited integer (e.g. a feature, element, characteristic, property, method/process step or limitation) or group of integers (e.g. features, elements, characteristics, properties, method/process steps or limitations) alone.

### A. Introduction

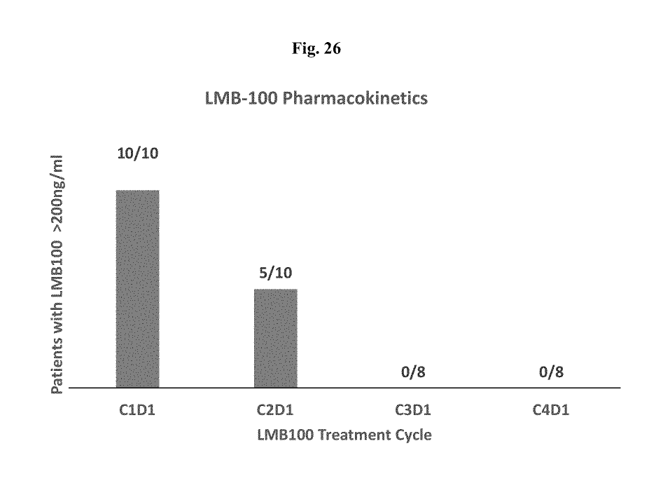

Recombinant immunotoxins (rITs), such as cancer targeting rITs, are potent therapeutics; however, such therapeutics can be very immunogenic and induce an immunogenicity response. The immunogenicity response can be characterized by the formation of anti-drug antibodies (ADAs) specific to the rIT, such as to the toxin of the rIT. The ADAs can limit the effectiveness of such therapies, even after one cycle of the therapy, and can cause severe hypersensitivity reactions in patients. The responses against the rIT can be so strong, for example, when the toxin is of bacterial origin, in most if not all patients, that treatment generally cannot progress. As an example, FIG. 26 illustrates how peak blood levels of LMB-100 after day 1 LMB-100 infusion during cycle 1 to 4 in subjects with mesothelioma. All subjects had LMB-100 blood levels during cycle 1, but the levels decreased to half during cycle 2, and no patients who received cycles 3 and 4 of treatment had desirable blood levels of the rIT. This illustrates the current inability to use such a rIT, such as over multiple treatment cycles, effectively without the benefit of the teachings provided herein.

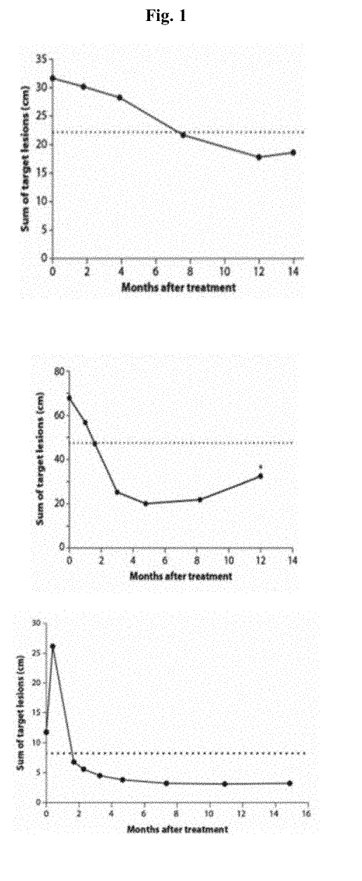

To combat the immunogenicity, some immunosuppressive therapies have been tried, although unsuccessfully. For example, FIG. 1, shows the highest overall mesothelioma tumor response in patients in the months following treatment with cyclophosphamide and pentostatin (CP/PS), an immunosuppressive therapy, and an immunotoxin, SS1(dsFv) PE38 (SS1P). However, the heavy immunosuppressive treatment only delayed the response to the immunotoxin, with the immunogenicity still limiting the number of treatment cycles with SS1P in most patients.

However, the inventors surprisingly found, however, that with the methods and composition provided herein, an unwanted immune response is not merely delayed but can be significantly reduced or eliminated long-term even in the subsequent absence of treatment with the synthetic nanocarriers comprising an immunosuppressant provided herein. In addition, it has been surprisingly found that the tolerance to the rIT can be achieved in a specific manner such that cancer growth is not promoted as a result of the immune modulation with the synthetic nanocarriers comprising an immunosuppressant. These results have not been achieved by immunosuppressive therapy, such as described above and shown in FIG. 1.

Thus, the discoveries made by the inventors can allow for long-term and repeated treatment with the rIT even when subsequently no immune modulating therapy is given, such as the synthetic nanocarriers comprising an immunosuppressant as provided herein. The methods and composition provided herein can create a neoplasia-neutral tolerogenic environment such that the immunogenicity against a rIT can be reduced or eliminated and treatment efficacy can be significantly improved long-term and/or with multiple cycles of treatment with the rIT (e.g., a least 2, 3, 4 or more treatment cycles).

As shown in the Examples, administration of synthetic nanocarriers comprising an immunosuppressant, such as rapamycin, with a rIT, such as LMB-100, was effective at inhibiting ADAs even upon subsequent LMB-100 challenges, both in the short-term (e.g., 2-3 weeks post-immunization) and long-term (e.g., 8 weeks post-immunization). The Examples also demonstrate the reduction in tumor size using methods and compositions provided herein as well as the lack of cancer growth promotion with an immunosuppressant as provided herein. Interestingly, tolerance induced by the synthetic nanocarriers comprising an immunosuppressant was not adversely affected by checkpoint inhibitors, an immune stimulator, such that the combination of a checkpoint inhibitor with the rIT and synthetic nanocarriers comprising an immunosuppressant can be contemplated for treatment. These results, however, are specific to the combination of the three agents together whereby ADA formation was not enhanced by the checkpoint inhibitor, and tumor size reduction was also demonstrated with the combination.

Additionally, it has been surprisingly found that the methods provided herein can be effective for subjects already undergoing an unwanted immune response against a rIT or previously having exposure to an immunogenic portion of the rIT, such as a bacterial toxin. For example, it was found that administration of synthetic nanocarriers comprising an immunosuppressant, such as rapamycin, reduced ADAs 3- to 7-fold in mice even with high preexisting anti-LMB-100 antibody titers (dilution >10,000). Thus, the synthetic nanocarriers comprising an immunosuppressant can be used as provided herein to mitigate the formation of inhibitory ADAs in naïve and in sensitized mice to a rIT, resulting in the restoration of anti-tumor activity. Accordingly, the subject of any one of the methods provided herein can be one with prior exposure to the rIT or an immunogenic portion thereof, such as a toxin or portion thereof. Any one of the subjects provided herein may be one who has already received treatment with the rIT or may be a treatment-naïve subject previously exposed to an immunogenic portion thereof in some other manner. Normally, without the methods and compositions provided herein, it would be expected that treatment with the rIT in such a subject would be largely ineffective.

Thus, provided herein are methods, and related compositions, for treating a subject with a cancer, for example, by creating a neoplasia-neutral tolerogenic environment in the subject as provided herein and administering a rIT to the subject in order to treat the cancer. As demonstrated within, such methods and compositions were found to inhibit or reduce unwanted immune responses and/or increase the efficacy of the rIT. The inventors have surprisingly and unexpectedly discovered that the problems and limitations noted above can be overcome by practicing the invention disclosed herein. Methods and compositions are provided that offer solutions to the aforementioned obstacles to immunogenicity and the use of rITs, such as for cancer treatment.

The invention will now be described in more detail below.

### B. Definitions

“Administering” or “administration” or “administer” means providing a material to a subject in a manner that is pharmacologically useful. The term includes causing to be administered. “Causing to be administered” means causing, urging, encouraging, aiding, inducing or directing, directly or indirectly, another party to administer the material.

“Amount effective” is any amount of a composition provided herein that results in one or more desired responses, such as one or more desired immune responses, including reduced immunogenicity against a rIT or an immunogenic portion of the rIT. This amount can be for in vitro or in vivo purposes. For in vivo purposes, the amount can be one that a clinician would believe may have a clinical benefit for a subject in need thereof, such as a subject that may experience undesired immune responses as a result of administration of a rIT. In any one of the methods provided herein, the compositions administered may be in any one of the amounts effective as provided herein.

Amounts effective can involve reducing the level of an undesired immune response, although in some embodiments, it involves preventing an undesired immune response altogether. Amounts effective can also involve delaying the occurrence of an undesired immune response. An amount effective can also be an amount that results in a desired therapeutic endpoint or a desired therapeutic result. Amounts effective, preferably, result in a tolerogenic immune response in a subject to a rIT. The achievement of any of the foregoing can be monitored by routine methods.

In one embodiment, the reduced immunogenicity persists in the subject. In still another embodiment, the reduced immunogenicity results or persists due to the administration of a composition provided herein according to a protocol or treatment regimen as provided herein. Amounts effective will depend, of course, on the particular subject being treated; the severity of a condition, disease or disorder; the individual patient parameters including age, physical condition, size and weight; the duration of the treatment; the nature of concurrent therapy (if any); the specific route of administration and like factors within the knowledge and expertise of the health practitioner. These factors are well known to those of ordinary skill in the art and can be addressed with no more than routine experimentation. It is generally preferred that a maximum dose be used, that is, the highest safe dose according to sound medical judgment. It will be understood by those of ordinary skill in the art, however, that a patient may insist upon a lower dose or tolerable dose for medical reasons, psychological reasons or for virtually any other reason.

In general, doses of the immunosuppressants and/or rITs in the compositions of the invention refer to the amount of the immunosuppressants and/or rITs. Alternatively, the dose can be administered based on the number of synthetic nanocarriers that provide the desired amount of immunosuppressant. Any one of the amounts of the immunosuppressants and/or rITs and/or synthetic nanocarriers of any one of the methods or compositions provided herein can be in an amount effective.

“Assessing an immune response” refers to any measurement or determination of the level, presence or absence, reduction in, increase in, etc. of an immune response in vitro or in vivo. Such measurements or determinations may be performed on one or more samples obtained from a subject. Such assessing can be performed with any of the methods provided herein or otherwise known in the art. The assessing may be assessing the number or percentage of antibodies or T cells, level of cytokine production, etc., such as in a sample from a subject.

“Average” refers to the mean unless indicated otherwise.

“Concomitantly” means administering two or more materials/agents to a subject in a manner that is correlated in time, preferably sufficiently correlated in time so as to provide a modulation in a physiologic or immunologic response, and even more preferably the two or more materials/agents are administered in combination. In embodiments, concomitant administration may encompass administration of two or more compositions within a specified period of time, preferably within 1 month, more preferably within 1 week, still more preferably within 1 day, and even more preferably within 1 hour. In embodiments, the compositions may be repeatedly administered concomitantly, that is concomitant administration on more than one occasion, such as may be provided herein.

In some embodiments of any one of the methods provided, the concomitant administration is “simultaneous”, which means that the administration is at the same time or substantially at the same time where a clinician would consider any time between administrations virtually nil or negligible as to the impact on the desired therapeutic outcome. In some embodiments of any one of the methods provided, the simultaneous administration is within 5 or fewer minutes of each other.

“Couple” or “Coupled” (and the like) means to chemically associate one entity (for example a moiety) with another. In some embodiments, the coupling is covalent, meaning that the coupling occurs in the context of the presence of a covalent bond between the two entities. In non-covalent embodiments, the non-covalent coupling is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. In embodiments, encapsulation is a form of coupling.

“Cycle” refers to an administration or set of administrations of an agent or agent(s) whereby there is expected to be some level of clinical benefit to the subject over the period of the administration or set of administrations. The end of a cycle of treatment occurs where there is a period of time with no administrations, preferably in an embodiment of any one of the methods provided herein, the end of a cycle of treatment occurs where there is a period of time with no expected additional significant clinical benefit seen in the subject after the period of the administration or set of administrations. In such embodiments, there is such a period of time between cycles. In an embodiment of any one of the methods provided herein, each cycle may be any one of the cycles of administration (e.g., dose and frequency of the rIT and/or synthetic nanocarriers comprising an immunosuppressant) provided herein including as described in the Examples.

“Creating” means causing an action to occur, either directly oneself or indirectly, such as, but not limited to, an unrelated third party that takes an action through reliance on one's words or deeds.

“Dosage form” means a pharmacologically and/or immunologically active material in a medium, carrier, vehicle, or device suitable for administration to a subject. Any one of the compositions or doses provided herein may be in a dosage form.

“Dose” refers to a specific quantity of a pharmacologically and/or immunologically active material for administration to a subject for a given time.

“Encapsulate” means to enclose at least a portion of a substance within a synthetic nanocarrier. In some embodiments, a substance is enclosed completely within a synthetic nanocarrier. In other embodiments, most or all of a substance that is encapsulated is not exposed to the local environment external to the synthetic nanocarrier. In other embodiments, no more than 50%, 40%, 30%, 20%, 10% or 5% (weight/weight) is exposed to the local environment. Encapsulation is distinct from absorption, which places most or all of a substance on a surface of a synthetic nanocarrier, and leaves the substance exposed to the local environment external to the synthetic nanocarrier.

“Identifying a subject” is any action or set of actions that allows a clinician to recognize a subject as one who may benefit from the methods or compositions provided herein or some other indicator as provided. Preferably, the identified subject is one who is in need of a tolerogenic immune response to a rIT. Such subjects include any subject that has or is at risk of having cancer. The action or set of actions may be either directly oneself or indirectly, such as, but not limited to, an unrelated third party that takes an action through reliance on one's words or deeds. In one embodiment of any one of the methods provided herein, the method further comprises identifying a subject in need of a composition or method as provided herein. In one embodiment of any one of the methods provided herein, the method further comprises identifying a subject in need of a neoplasia-neutral tolerogenic environment as provided herein.

“Immune checkpoint inhibitor” is any molecule that directly or indirectly inhibits, partially or completely, an immune checkpoint pathway. Aspects of the disclosure are related to the observation that inhibiting such immune checkpoint pathways in combination with synthetic nanocarriers comprising an immunosuppressant and a rIT can still result in a reduction in immunogenicity to the rIT and/or improved treatment efficacy as compared to the rIT alone in the presence of an ADA response. Examples of immune checkpoint pathways include, without limitation, PD-1/PD-L1, CTLA4/B7-1, TIM-3, LAG3, By-He, H4, HAVCR2, IDO1, CD276 and VTCN1 as well as monoclonal antibodies, such as BMS-936558/MDX-1106, BMS-936559/MDX-1105, ipilimumab/Yervoy, and tremelimumab; humanized antibodies, such as CT-011 and MK-3475; and fusion proteins, such as AMP-224, and the antibodies of the Examples.

“Immunosuppressant” means a compound that can cause a tolerogenic effect, preferably through its effects on APCs. A tolerogenic effect generally refers to the modulation by the APC or other immune cells that reduces, inhibits or prevents an undesired immune response to an antigen in a durable fashion. In one embodiment of any one of the methods or compositions provided, the immunosuppressant is one that causes an APC to promote a regulatory phenotype in one or more immune effector cells. For example, the regulatory phenotype may be characterized by the inhibition of the production, induction, stimulation or recruitment of antigen-specific CD4+ T cells or B cells, the inhibition of the production of antigen-specific antibodies, the production, induction, stimulation or recruitment of Treg cells (e.g., CD4+CD25highFoxP3+ Treg cells), etc. This may be the result of the conversion of CD4+ T cells or B cells to a regulatory phenotype. This may also be the result of induction of FoxP3 in other immune cells, such as CD8+ T cells, macrophages and iNKT cells. In one embodiment of any one of the methods or compositions provided, the immunosuppressant is one that affects the response of the APC after it processes an antigen. In another embodiment of any one of the methods or compositions provided, the immunosuppressant is not one that interferes with the processing of the antigen. In a further embodiment of any one of the methods or compositions provided, the immunosuppressant is not an apoptotic-signaling molecule. In another embodiment of any one of the methods or compositions provided, the immunosuppressant is not a phospholipid.

Immunosuppressants include, but are not limited to mTOR inhibitors, such as rapamycin or a rapamycin analog (i.e., rapalog); TGF-β signaling agents; TGF-β receptor agonists; histone deacetylase inhibitors, such as Trichostatin A; corticosteroids; inhibitors of mitochondrial function, such as rotenone; P38 inhibitors; NF-κβ inhibitors, such as 6Bio, Dexamethasone, TCPA-1, IKK VII; adenosine receptor agonists; prostaglandin E2 agonists (PGE2), such as Misoprostol; phosphodiesterase inhibitors, such as phosphodiesterase 4 inhibitor (PDE4), such as Rolipram; proteasome inhibitors; kinase inhibitors; etc. “Rapalog”, as used herein, refers to a molecule that is structurally related to (an analog) of rapamycin (sirolimus). Examples of rapalogs include, without limitation, temsirolimus (CCI-779), everolimus (RAD001), ridaforolimus (AP-23573), and zotarolimus (ABT-578). Additional examples of rapalogs may be found, for example, in WO Publication WO 1998/002441 and U.S. Pat. No. 8,455,510, the rapalogs of which are incorporated herein by reference in their entirety. Further immunosuppressants are known to those of skill in the art, and the invention is not limited in this respect.

In embodiments, when coupled to the synthetic nanocarriers, the immunosuppressant is an element that is in addition to the material that makes up the structure of the synthetic nanocarrier. For example, in one such embodiment, where the synthetic nanocarrier is made up of one or more polymers, the immunosuppressant is a compound that is in addition and coupled to the one or more polymers. As another example, in one such embodiment, where the synthetic nanocarrier is made up of one or more lipids, the immunosuppressant is again in addition and coupled to the one or more lipids. In another of such embodiments, such as where the material of the synthetic nanocarrier also results in a tolerogenic effect, the immunosuppressant is an element present in addition to the material of the synthetic nanocarrier that results in a tolerogenic effect.

“Load”, when coupled to a synthetic nanocarrier, is the amount of the immunosuppressant coupled to the synthetic nanocarrier based on the total dry recipe weight of materials in an entire synthetic nanocarrier (weight/weight). Generally, such a load is calculated as an average across a population of synthetic nanocarriers. In one embodiment of any one of the methods or compositions provided, the load on average across the synthetic nanocarriers is between 0.1% and 50%. In another embodiment of any one of the methods or compositions provided, the load is between 0.1% and 20%. In a further embodiment of any one of the methods or compositions provided, the load is between 0.1% and 10%. In still a further embodiment of any one of the methods or compositions provided, the load is between 1% and 10%. In still a further embodiment of any one of the methods or compositions provided, the load is between 7% and 20%. In yet another embodiment of any one of the methods or compositions provided, the load is at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5%, at least 6%, at least at least 7%, at least 8%, at least 9%, at least 10%, at least 11%, at least 12%, at least 13%, at least 14%, at least 15%, at least 16%, at least 17%, at least 18%, at least 19% or at least 20% on average across the population of synthetic nanocarriers. In yet a further embodiment of any one of the methods or compositions provided, the load is 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19% or 20% on average across the population of synthetic nanocarriers. In some embodiments of any one of the above embodiments, the load is no more than 25% on average across a population of synthetic nanocarriers. In embodiments of any one of the methods or compositions provided, the load is calculated as known in the art.

“Maximum dimension of a synthetic nanocarrier” means the largest dimension of a nanocarrier measured along any axis of the synthetic nanocarrier. “Minimum dimension of a synthetic nanocarrier” means the smallest dimension of a synthetic nanocarrier measured along any axis of the synthetic nanocarrier. For example, for a spheroidal synthetic nanocarrier, the maximum and minimum dimension of a synthetic nanocarrier would be substantially identical, and would be the size of its diameter. Similarly, for a cuboidal synthetic nanocarrier, the minimum dimension of a synthetic nanocarrier would be the smallest of its height, width or length, while the maximum dimension of a synthetic nanocarrier would be the largest of its height, width or length. In an embodiment, a minimum dimension of at least 75%, preferably at least 80%, more preferably at least 90%, of the synthetic nanocarriers in a sample, based on the total number of synthetic nanocarriers in the sample, is equal to or greater than 100 nm. In an embodiment, a maximum dimension of at least 75%, preferably at least 80%, more preferably at least 90%, of the synthetic nanocarriers in a sample, based on the total number of synthetic nanocarriers in the sample, is equal to or less than 5 μm. Preferably, a minimum dimension of at least 75%, preferably at least 80%, more preferably at least 90%, of the synthetic nanocarriers in a sample, based on the total number of synthetic nanocarriers in the sample, is greater than 110 nm, more preferably greater than 120 nm, more preferably greater than 130 nm, and more preferably still greater than 150 nm. Aspects ratios of the maximum and minimum dimensions of inventive synthetic nanocarriers may vary depending on the embodiment. For instance, aspect ratios of the maximum to minimum dimensions of the synthetic nanocarriers may vary from 1:1 to 1,000,000:1, preferably from 1:1 to 100,000:1, more preferably from 1:1 to 10,000:1, more preferably from 1:1 to 1000:1, still more preferably from 1:1 to 100:1, and yet more preferably from 1:1 to 10:1. Preferably, a maximum dimension of at least 75%, preferably at least 80%, more preferably at least 90%, of the synthetic nanocarriers in a sample, based on the total number of synthetic nanocarriers in the sample is equal to or less than 3 μm, more preferably equal to or less than 2 μm, more preferably equal to or less than 1 μm, more preferably equal to or less than 800 nm, more preferably equal to or less than 600 nm, and more preferably still equal to or less than 500 nm. In preferred embodiments, a minimum dimension of at least 75%, preferably at least 80%, more preferably at least 90%, of the synthetic nanocarriers in a sample, based on the total number of synthetic nanocarriers in the sample, is equal to or greater than 100 nm, more preferably equal to or greater than 120 nm, more preferably equal to or greater than 130 nm, more preferably equal to or greater than 140 nm, and more preferably still equal to or greater than 150 nm. Measurement of synthetic nanocarrier dimensions (e.g., diameter) may be obtained by suspending the synthetic nanocarriers in a liquid (usually aqueous) media and using dynamic light scattering (DLS) (e.g. using a Brookhaven ZetaPALS instrument). For example, a suspension of synthetic nanocarriers can be diluted from an aqueous buffer into purified water to achieve a final synthetic nanocarrier suspension concentration of approximately 0.01 to 0.1 mg/mL. The diluted suspension may be prepared directly inside, or transferred to, a suitable cuvette for DLS analysis. The cuvette may then be placed in the DLS, allowed to equilibrate to the controlled temperature, and then scanned for sufficient time to acquire a stable and reproducible distribution based on appropriate inputs for viscosity of the medium and refractive indicies of the sample. The effective diameter, or mean of the distribution, can then reported. “Dimension” or “size” or “diameter” of synthetic nanocarriers means the mean of a particle size distribution obtained using dynamic light scattering in some embodiments.

“Mesothelin-expressing cancer” refers to any cancer with cells that express mesothelin. Mesothelin, generally considered a 40 kDa GPI-linked glycoprotein antigen, is found on the surface of mesothelial cells and is expressed on solid tumors, including those associated with the lung, pleura, ovary, breast, stomach, bile ducts, uterus, and thymus (Pastan et al., Cancer Res. 2014; 74: 2907-2912). Thus, examples of mesothelin-expressing cancers include, but are not limited to, mesothelioma, pancreatic adenocarcinoma, ovarian cancer, lung adenocarcinoma, breast cancer, and gastric cancer, as well as any of those immediately above.

“Neoplasia-neutral tolerogenic environment” refers to creating an environment whereby an unwanted immune response against a rIT used to treat the cancer is reduced or eliminated while the immune reduction does not result in promotion of cancer growth. Generally, once this environment has been created an unwanted immune response in reduced or eliminated even when the rIT is administered alone. In an embodiment of any one of the methods provided herein, creating such an environment allows for treatment with a rIT long-term and/or that includes multiple administrations (e.g., at least 2, 3, 4 or more) or treatment cycles (e.g., at least 2, 3, 4 or more).

“Non-hematologic cancers” are those that do not begin in the blood or bone marrow and are known in the art. Such cancers include, but are not limited to, brain cancer, cancers of the head and neck, lung cancer, breast cancer, cancers of the reproductive system, cancers of the gastro-intestinal system, pancreatic cancer, and cancers of the urinary system, cancer of the upper digestive tract or colorectal cancer, bladder cancer or renal cell carcinoma, and prostate cancer.

“Pharmaceutically acceptable excipient” or “pharmaceutically acceptable carrier” means a pharmacologically inactive material used together with an active material to formulate the compositions. Pharmaceutically acceptable excipients or carriers comprise a variety of materials known in the art, including but not limited to saccharides (such as glucose, lactose, and the like), preservatives such as antimicrobial agents, reconstitution aids, colorants, saline (such as phosphate buffered saline), and buffers.

“Protocol” refers to any dosing regimen of one or more substances to a subject. A dosing regimen may include the amount, frequency, rate, duration and/or mode of administration. In some embodiments, such a protocol may be used to administer one or more compositions of the invention to one or more test subjects. Immune responses in these test subjects can then be assessed to determine whether or not the protocol was effective in generating a desired immune response, such as a tolerogenic immune response against a rIT. Any other therapeutic and/or prophylactic effects may also be assessed instead of or in addition to the aforementioned immune responses. Whether or not a protocol had a desired effect can be determined using any of the methods provided herein or otherwise known in the art. For example, a population of cells may be obtained from a subject to which a composition provided herein has been administered according to a specific protocol in order to determine whether or not specific immune cells, cytokines, antibodies, etc. were generated, activated, etc. Useful methods for detecting the presence and/or number of immune cells include, but are not limited to, flow cytometric methods (e.g., FACS) and immunohistochemistry methods. Antibodies and other binding agents for specific staining of immune cell markers, are commercially available. Such kits typically include staining reagents for multiple antigens that allow for FACS-based detection, separation and/or quantitation of a desired cell population from a heterogeneous population of cells. Any one of the methods provided herein can include a step of determining a protocol and/or the administering is done based on a protocol determined to have any one of the beneficial results as provided herein.

“Recombinant immunotoxin” means a compound for treatment, such as cancer treatment, of a subject that comprises a ligand and a toxin. In some embodiments, when the rIT is administered to a subject without synthetic nanocarriers comprising an immunosuppressant, the rIT generates, or is expected to generate, an unwanted immune response, such as unwanted antibodies against the rIT. In some embodiments, the rIT comprises an antibody, or antigen binding fragment thereof, and a toxin. In some embodiments, the rIT is LMB-100. In an embodiment, the rIT of any one of the methods or compositions provided herein is one where a neoplasia-neutral environment is needed in order for treatment in a subject to be efficacious. Such a rIT is generally one where the toxin is quite immunogenic. Such rITs include those that comprise a toxin of bacterial origin, a plant toxin, or a venom toxin, such as one of an insect. Other examples would be known in the art or otherwise provided herein. Any one of the rITs provided herein may be the rIT of any one of the methods or compositions provided herein.

“Recombinant immunotoxin immune response” refers to any immune response against a rIT. Generally, such immune responses are undesired or unwanted and can interfere with the therapeutic efficacy of the rIT. Accordingly, the immune response can be specific to the rIT, which refers to an immune response that results from the presence of the rIT or portion thereof, such as the toxin or portion thereof. Generally, while such responses are measurable against the rIT or portion thereof, the responses are reduced or negligible in regard to other antigens. In some embodiments of any one of the methods or compositions provided herein, the immune response to the rIT or portion thereof is an antibody immune response as provided herein.

“Similar level” refers to a level of a response that a person of skill in the art would expect to be a comparable result. Similar responses in some embodiments are not considered to be statistically different. Whether or not a similar response is generated can be determined with in vitro or in vivo techniques. For example, whether or not a similar level of cell killing is generated can be determined by determining an IC50 level in vitro. As another example, assessment of in vitro cytotoxicity of rITs can be undertaken by contacting rIT with target cells in 96 well plates and analyzed 24-96 hours later. Quantification of cell death can be accomplished by determining the uptake of 3H-thymidine by surviving cells. Specificity can be determined by use of control cells, blocking with excess unlabeled antibody, or control rITs.

As another example, whether or not a similar level of efficacy, such as therapeutic efficacy, is generated can be determined by a variety of techniques measuring any indicator of such efficacy. Such indicators can be measured in animal or clinical trial subjects, and the subjects to which the compositions are administered according to the methods provided herein can be the same or different. For example, a mouse can be used to determine the effect of a rIT on tumor size. Animal survival rates may also be determined. Other indicators of efficacy include a decrease in the number of cancer cells, a decrease in the level of a biomarker indicative of the presence of cancer cells in serum, the onset or decrease in symptoms, such as bone pain, the onset or increase in metastases, etc. Assays and techniques for assessing indicators of efficacy, such as therapeutic efficacy, are known in the art.

“Subject” means animals, including warm blooded mammals such as humans and primates; avians; domestic household or farm animals such as cats, dogs, sheep, goats, cattle, horses and pigs; laboratory animals such as mice, rats and guinea pigs; fish; reptiles; zoo and wild animals; and the like.

“Synthetic nanocarrier(s)” means a discrete object that is not found in nature, and that possesses at least one dimension that is less than or equal to 5 microns in size. Albumin nanoparticles are generally included as synthetic nanocarriers, however in certain embodiments the synthetic nanocarriers do not comprise albumin nanoparticles. In embodiments, synthetic nanocarriers do not comprise chitosan. In certain other embodiments, the synthetic nanocarriers do not comprise chitosan. In other embodiments, inventive synthetic nanocarriers are not lipid-based nanoparticles. In further embodiments, inventive synthetic nanocarriers do not comprise a phospholipid.

A synthetic nanocarrier can be, but is not limited to, one or a plurality of lipid-based nanoparticles (also referred to herein as lipid nanoparticles, i.e., nanoparticles where the majority of the material that makes up their structure are lipids), polymeric nanoparticles, metallic nanoparticles, surfactant-based emulsions, dendrimers, buckyballs, nanowires, virus-like particles (i.e., particles that are primarily made up of viral structural proteins but that are not infectious or have low infectivity), peptide or protein-based particles (also referred to herein as protein particles, i.e., particles where the majority of the material that makes up their structure are peptides or proteins) (such as albumin nanoparticles) and/or nanoparticles that are developed using a combination of nanomaterials such as lipid-polymer nanoparticles. Synthetic nanocarriers may be a variety of different shapes, including but not limited to spheroidal, cuboidal, pyramidal, oblong, cylindrical, toroidal, and the like. Synthetic nanocarriers according to the invention comprise one or more surfaces. Exemplary synthetic nanocarriers that can be adapted for use in the practice of the present invention comprise: (1) the biodegradable nanoparticles disclosed in U.S. Pat. No. 5,543,158 to Gref et al., (2) the polymeric nanoparticles of Published US Patent Application 20060002852 to Saltzman et al., (3) the lithographically constructed nanoparticles of Published US Patent Application 20090028910 to DeSimone et al., (4) the disclosure of WO 2009/051837 to von Andrian et al., (5) the nanoparticles disclosed in Published US Patent Application 2008/0145441 to Penades et al., (6) the protein nanoparticles disclosed in Published US Patent Application 20090226525 to de los Rios et al., (7) the virus-like particles disclosed in published US Patent Application 20060222652 to Sebbel et al., (8) the nucleic acid coupled virus-like particles disclosed in published US Patent Application 20060251677 to Bachmann et al., (9) the virus-like particles disclosed in WO2010047839A1 or WO2009106999A2, (10) the nanoprecipitated nanoparticles disclosed in P. Paolicelli et al., “Surface-modified PLGA-based Nanoparticles that can Efficiently Associate and Deliver Virus-like Particles” Nanomedicine. 5(6):843-853 (2010), (11) apoptotic cells, apoptotic bodies or the synthetic or semisynthetic mimics disclosed in U.S. Publication 2002/0086049, or (12) those of Look et al., Nanogel-based delivery of mycophenolic acid ameliorates systemic lupus erythematosus in mice” J. Clinical Investigation 123(4):1741-1749(2013).

Synthetic nanocarriers according to the invention that have a minimum dimension of equal to or less than about 100 nm, preferably equal to or less than 100 nm, do not comprise a surface with hydroxyl groups that activate complement or alternatively comprise a surface that consists essentially of moieties that are not hydroxyl groups that activate complement. In a preferred embodiment, synthetic nanocarriers according to the invention that have a minimum dimension of equal to or less than about 100 nm, preferably equal to or less than 100 nm, do not comprise a surface that substantially activates complement or alternatively comprise a surface that consists essentially of moieties that do not substantially activate complement. In a more preferred embodiment, synthetic nanocarriers according to the invention that have a minimum dimension of equal to or less than about 100 nm, preferably equal to or less than 100 nm, do not comprise a surface that activates complement or alternatively comprise a surface that consists essentially of moieties that do not activate complement. In embodiments, synthetic nanocarriers may possess an aspect ratio greater than 1:1, 1:1.2, 1:1.5, 1:2, 1:3, 1:5, 1:7, or greater than 1:10.

“Therapeutic efficacy” refers to any of the desired effects of a treatment, such as with a rIT. Such effects include the inhibition in the onset or progression of a disease, such as cancer, or a symptom thereof. Other examples of indicators of therapeutic efficacy are provided elsewhere herein or would be otherwise apparent to one of ordinary skill in the art.

### C. Compositions and Related Methods

The development of anti-drug antibodies (ADAs) limits the effectiveness of therapies, such as rITs and can cause severe hypersensitivity reactions in patients. The formation of ADAs has been a limiting factor in the clinical efficacy of, for example, rITs for cancer therapy. A large majority of immune-competent patients develop neutralizing anti-rIT antibodies after one cycle of treatment, which reduces anti-cancer efficacy and prohibits further treatment. Prior exposure to a toxin, such as that of P. aeruginosa, is one mechanism whereby treatment-naïve patients could present with pre-existing antibodies against exotoxin A, making even the first cycle of rIT treatment ineffective. Provided herein are compositions and methods for reducing unwanted immune responses to such rITs, thereby increasing the efficacy of the rIT, such as in the treatment of cancer. It has been found that through creating a neoplasia-neutral tolerogenic environment, such as with the administration of synthetic nanocarriers comprising an immunosuppressant, such as rapamycin, the immunogenicity of a rIT can be reduced and the efficacy of the rIT increased through rounds or cycles of administration (and/or even allowing multiple rounds or cycles of administration).

In some embodiments, the rIT can target cancer cells, such as via an antigen expressed thereby or thereon. Cancer antigens can be associated with or characteristic of only one type of cancer. Cancer antigens, however, can be associated with or characteristic of more than one type of cancer. Examples of cancer antigens include, but are not limited to, mesothelin, CD5, CD7, CD19, CD20, CD22, CD25, CD30, CD33, CD52, CD56, CD66, EpCAM, CEA, gpA33, mucins, MAGE (melanoma associated antigen), PRAME (preferentially expressed antigen of melanoma), TAG-72, carbonic anhydrase IX, PSMA, tyrosinase tumor antigen, NY-ESO-1, telomerase, p53, folate binding protein, gangliosides (GD2, GD3, GM, etc.), Lewis-Y antigen (a carbohydrate antigen), IL2R, IL4R, IL13R, TfR (transferrin receptor), GM-CSFR, ErbB1/EGFR, ErbB2/HER2, ErbB3, c-Met, IGF1R, EGFR, mutant epidermal growth factor receptor variant III, VEGF, VEGFR, αVβ3, α5β1, GPNMB (glycoprotein non-metastatic melanoma protein B), HMW-MAA (high molecular weight melanoma-associated antigen), EphA2, EphA3, uPAR (urokinase-type plasminogen activator receptor), proteoglycan, TRAIL-R1, TRAIL-R2, RANKL, FAP, and tenascin.

The ligand of the rIT may be any targeting molecule. For example, the ligand may be an antibody, an antibody fragment, such as a single-chain antibody, or a natural ligand, such as a cytokine, a growth factor, or a peptide hormone (Weng et al., Mol Oncol. 2012, 6(3): 323-332). If the targeting ligand is an antibody or antigen-binding fragment thereof, it may be monoclonal or recombinant, including chimeras or variable region fragments.

As used herein, “antibody” refers to a glycoprotein comprising at least two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FRs). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

As used herein, “antigen-binding fragment” of an antibody refers to one or more portions of an antibody that retain the ability to bind specifically to an antigen. The antigen-binding function of an antibody can be performed by fragments of a full-length antibody. Examples of binding fragments encompassed within the term “antigen-binding fragment” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, V and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional procedures, such as proteolytic fragmentation procedures, as described in J. Goding, Monoclonal Antibodies: Principles and Practice, pp 98-118 (N.Y. Academic Press 1983), which is hereby incorporated by reference, as well as by other techniques known to those with skill in the art. The fragments are screened for utility in the same manner as are intact antibodies.

Any toxin may be conjugated to a ligand as provided herein to form a rIT. In some embodiments, the ligand and toxin are covalently linked. Toxins may come from or be based on a variety of sources, including plants, insects, vertebrates, bacteria, and fungi. Examples of toxins include, but are not limited to, Pseudomonas aeruginosa exotoxin A (PE), diphtheria toxin (DT) from Corynebacterium diphtheria, saponin from Saponaria officinalis, shiga toxin, abrin from Abrus precatorius seeds, dianthin-30, ricin-A-chain (RTA), pokeweed antiviral protein (PAP), gelonin, bryodin 1, calicheamicin toxin, etc.

In some embodiments, the toxin is of bacterial origin or based on such a toxin, and may be, for example, a bacterial toxin, such as Pseudomonas aeruginosa exotoxin A (PE), Pseudomonas aeruginosa endotoxin, diphtheria toxin (DT; Corynebacterium diphtheria, Clostridium perfringens enterotoxin (CPE), alpha toxin (for example, from Staphylococcus aureus, Clostridium perfringens, or Pseudomonas aeruginosa), Staphylococcal enterotoxin-A, α-sarcin (Aspergillus giganteus), Shiga toxin (for example, from Escherichia coli or Shigella dysenteriae), calicheamicin toxin (Micromonospora echinospora), and cyclomodulins (such as, cytotoxin necrotizing factor, CNF).

In some embodiments, the toxin may be of a plant source or based on such a toxin, and may be, for example, a plant toxin, such as holotoxin (e.g., class I ribosome-inactivating proteins) or a hemitoxin (e.g., class II ribosome-inactivating proteins). Examples of holotoxins include, but are not limited to, ricin, abrin, modeccin, and mistletoe lectin. Examples of hemitoxins include, but are not limited to, pokeweed antiviral protein (PAP), gelonin, saporin, bouganin, and bryodin.

The toxin may also be from or based on fungi. Examples of fungal toxins include, but are not limited to, aspergillin and restrictocin.

In some embodiments, the toxin is a venom toxin, and may be, for example from or based on an insect toxin. Examples of insect toxins which may be used include, but are not limited to, mastoparans (MPs) (Polybia-MP1, Polybia-MPII, and Polybia-MPIII from Polybia paulista), 7,8-seco-para-ferruginone (SPF; from Vespa simillima), melittin (from Apis mellifera), and phospholipase A2 (PLA2; from Apis mellifera).

Examples of rITs include any one of the toxins provided herein (or a portion thereof). Additional examples of rITs include those that are rITs for treating solid tumors as well as rITs for treating hematological cancers. Further examples of rITs include, but are not limited to, inotuzumab ozogamicin (a humanized anti-CD22 antibody and ozogamicin, a calicheamicin), moxetumomab pasudotox (an anti-CD22 monoclonal antibody and PE38, a 38 kDa fragment of Psuedomonas exotoxin A), LMB-2 (an anti-CD25 o IL-24 antibody and PE38), and VB4-845 (an anti-EpCAM single-chain antibody fragment and PE38). Further examples of rITs include: LMB-1, LMB-7, LMB-9, BL22/CAT-3888, SS1P (SS1(dsFv)-PE38), DT388-IL3, HA22/CAT-8015, deglycosylated ricin A chain-conjugated anti-CD19/Anti-CD22, DT2219, D2C7-IT, A-dmDT390-bisFv(UCHT1), AB389IL2, DT388 GMCSF, RFB4-dgA, HD37-dgA, Combotox (RFB4-dgA+HD37-dgA), RFTS-dgA (IMTOX-25), Ki-4.dgA, HuM195/rGel, Erb38, scFv(FRPS)-ETA, SGN-10, OvB3-PE, TP40, D2C7-(scdsFv)-PE38KDEL (D2C7-IT), MR1(Fv)-PE38 (MR1), MR1-1(Fv)-PE38 (MR1-1), TGFα-PE38 (TP38), TGFα-PE40 (TP40), DAB389EGF, DT390-BiscFv806, ScFv(14E1)-ETA, Anti-EGFR/LP1, IL-13PE38QQR (IL-13PE), IL13E13K-PE38, Anti-IL-13Ra2(scFv)-PE38, DT3901L13, IL4(38-37)-PE38KDEL (cpIL4-PE), DT390-mIL4, DT390-ATF (DTAT), DT390-IL-13-ATF (DTAT13), EGFATFKDEL, EGFATFKDEL7mut, DTEGF13, 8H9scFv-PE38, EphrinA1-PE38QQR, NZ-1-(scdsFv)-PE38KDEL, DmAb14m-(scFv)-PE38KDEL (DmAb14m-IT), and IT-87.

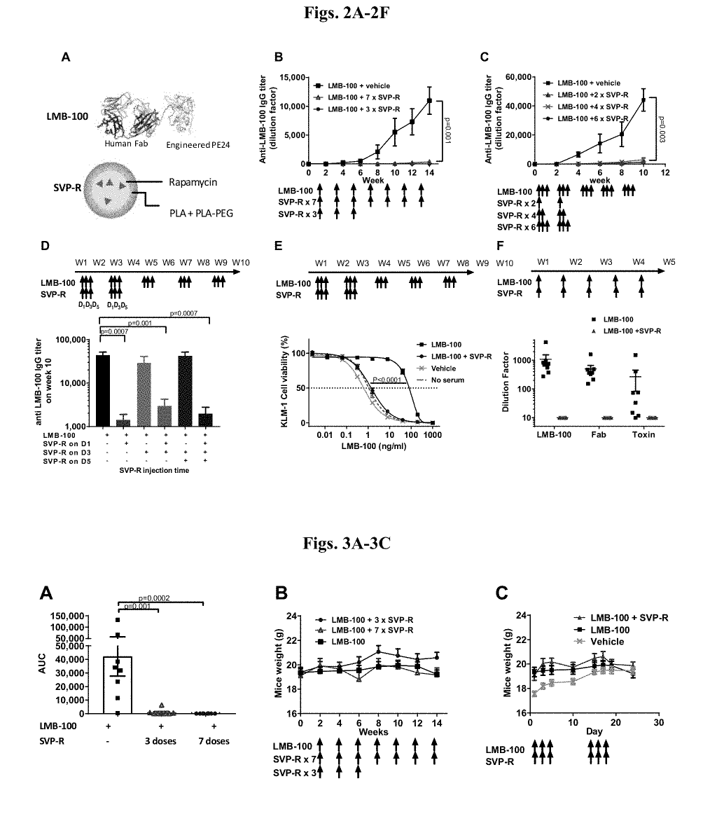

In some embodiments, the rIT is one with a tumor antigen-targeting antibody variable domain (Fv) that is linked, for example, covalently, to a toxin, such as one of bacterial origin (e.g., a domain of Pseudomonas aeruginosa exotoxin A). Thus, in any one of the methods or compositions provided herein, the rIT can be LMB-100, a second generation rIT that comprises a humanized Fab targeting mesothelin fused to a modified toxin (FIG. 2A). Additional rITs useful in accordance with aspects of this invention will be apparent to those of skill in the art, and the invention is not limited in this respect.

The methods provided herein include administrations of synthetic nanocarriers comprising an immunosuppressant. Generally, the immunosuppressant is an element that is in addition to the material that makes up the structure of the synthetic nanocarrier. For example, in one embodiment of any one of the methods or compositions provided, where the synthetic nanocarrier is made up of one or more polymers, the immunosuppressant is a compound that is in addition and, in some embodiments of any one of the methods or compositions provided, attached to the one or more polymers. In embodiments where the material of the synthetic nanocarrier also results in a tolerogenic effect, the immunosuppressant is an element present in addition to the material of the synthetic nanocarrier that results in a tolerogenic effect.

A wide variety of other synthetic nanocarriers can be used according to the invention, and in some embodiments of any one of the methods or compositions provided, coupled to an immunosuppressant. In some embodiments, synthetic nanocarriers are spheres or spheroids. In some embodiments, synthetic nanocarriers are flat or plate-shaped. In some embodiments, synthetic nanocarriers are cubes or cubic. In some embodiments, synthetic nanocarriers are ovals or ellipses. In some embodiments, synthetic nanocarriers are cylinders, cones, or pyramids.

In some embodiments of any one of the methods or compositions provided, it is desirable to use a population of synthetic nanocarriers that is relatively uniform in terms of size or shape so that each synthetic nanocarrier has similar properties. For example, at least 80%, at least 90%, or at least 95% of the synthetic nanocarriers of any one of the compositions or methods provided, based on the total number of synthetic nanocarriers, may have a minimum dimension or maximum dimension that falls within 5%, 10%, or 20% of the average diameter or average dimension of the synthetic nanocarriers.

Synthetic nanocarriers can be solid or hollow and can comprise one or more layers. In some embodiments, each layer has a unique composition and unique properties relative to the other layer(s). To give but one example, synthetic nanocarriers may have a core/shell structure, wherein the core is one layer (e.g. a polymeric core) and the shell is a second layer (e.g. a lipid bilayer or monolayer). Synthetic nanocarriers may comprise a plurality of different layers.

In some embodiments, synthetic nanocarriers may optionally comprise one or more lipids. In some embodiments, a synthetic nanocarrier may comprise a liposome. In some embodiments, a synthetic nanocarrier may comprise a lipid bilayer. In some embodiments, a synthetic nanocarrier may comprise a lipid monolayer. In some embodiments, a synthetic nanocarrier may comprise a micelle. In some embodiments, a synthetic nanocarrier may comprise a core comprising a polymeric matrix surrounded by a lipid layer (e.g., lipid bilayer, lipid monolayer, etc.). In some embodiments, a synthetic nanocarrier may comprise a non-polymeric core (e.g., metal particle, quantum dot, ceramic particle, bone particle, viral particle, proteins, nucleic acids, carbohydrates, etc.) surrounded by a lipid layer (e.g., lipid bilayer, lipid monolayer, etc.).

In other embodiments, synthetic nanocarriers may comprise metal particles, quantum dots, ceramic particles, etc. In some embodiments, a non-polymeric synthetic nanocarrier is an aggregate of non-polymeric components, such as an aggregate of metal atoms (e.g., gold atoms).

In some embodiments of any one of the methods or compositions provided, synthetic nanocarriers can comprise one or more polymers. In some embodiments of any one of the methods or compositions provided, the synthetic nanocarriers comprise one or more polymers that is a non-methoxy-terminated, pluronic polymer. In some embodiments of any one of the methods or compositions provided, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, or 99% (weight/weight) of the polymers that make up the synthetic nanocarriers are non-methoxy-terminated, pluronic polymers. In some embodiments of any one of the methods or compositions provided, all of the polymers that make up the synthetic nanocarriers are non-methoxy-terminated, pluronic polymers. In some embodiments of any one of the methods or compositions provided, the synthetic nanocarriers comprise one or more polymers that is a non-methoxy-terminated polymer. In some embodiments of any one of the methods or compositions provided, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, or 99% (weight/weight) of the polymers that make up the synthetic nanocarriers are non-methoxy-terminated polymers. In some embodiments of any one of the methods or compositions provided, all of the polymers that make up the synthetic nanocarriers are non-methoxy-terminated polymers. In some embodiments of any one of the methods or compositions provided, the synthetic nanocarriers comprise one or more polymers that do not comprise pluronic polymer. In some embodiments of any one of the methods or compositions provided, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, or 99% (weight/weight) of the polymers that make up the synthetic nanocarriers do not comprise pluronic polymer. In some embodiments of any one of the methods or compositions provided, all of the polymers that make up the synthetic nanocarriers do not comprise pluronic polymer. In some embodiments of any one of the methods or compositions provided, such a polymer can be surrounded by a coating layer (e.g., liposome, lipid monolayer, micelle, etc.). In some embodiments of any one of the methods or compositions provided, elements of the synthetic nanocarriers can be attached to the polymer.

Immunosuppressants can be coupled to the synthetic nanocarriers by any of a number of methods. Generally, the attaching can be a result of bonding between the immunosuppressants and the synthetic nanocarriers. This bonding can result in the immunosuppressants being attached to the surface of the synthetic nanocarriers and/or contained (encapsulated) within the synthetic nanocarriers. In some embodiments of any one of the methods or compositions provided, however, the immunosuppressants are encapsulated by the synthetic nanocarriers as a result of the structure of the synthetic nanocarriers rather than bonding to the synthetic nanocarriers. In preferable embodiments of any one of the methods or compositions provided, the synthetic nanocarrier comprises a polymer as provided herein, and the immunosuppressants are coupled to the polymer.

When coupling occurs as a result of bonding between the immunosuppressants and synthetic nanocarriers, the coupling may occur via a coupling moiety. A coupling moiety can be any moiety through which an immunosuppressant is bonded to a synthetic nanocarrier. Such moieties include covalent bonds, such as an amide bond or ester bond, as well as separate molecules that bond (covalently or non-covalently) the immunosuppressant to the synthetic nanocarrier. Such molecules include linkers or polymers or a unit thereof. For example, the coupling moiety can comprise a charged polymer to which an immunosuppressant electrostatically binds. As another example, the coupling moiety can comprise a polymer or unit thereof to which it is covalently bonded.

In preferred embodiments of any one of the methods or compositions provided, the synthetic nanocarriers comprise a polymer as provided herein. These synthetic nanocarriers can be completely polymeric or they can be a mix of polymers and other materials.

In some embodiments of any one of the methods or compositions provided, the polymers of a synthetic nanocarrier associate to form a polymeric matrix. In some of these embodiments of any one of the methods or compositions provided, a component, such as an immunosuppressant, can be covalently associated with one or more polymers of the polymeric matrix. In some embodiments of any one of the methods or compositions provided, covalent association is mediated by a linker. In some embodiments of any one of the methods or compositions provided, a component can be non-covalently associated with one or more polymers of the polymeric matrix. For example, in some embodiments of any one of the methods or compositions provided, a component can be encapsulated within, surrounded by, and/or dispersed throughout a polymeric matrix. Alternatively or additionally, a component can be associated with one or more polymers of a polymeric matrix by hydrophobic interactions, charge interactions, van der Waals forces, etc. A wide variety of polymers and methods for forming polymeric matrices therefrom are known conventionally.

Polymers may be natural or unnatural (synthetic) polymers. Polymers may be homopolymers or copolymers comprising two or more monomers. In terms of sequence, copolymers may be random, block, or comprise a combination of random and block sequences. Typically, polymers in accordance with the present invention are organic polymers.

In some embodiments of any one of the methods or compositions provided, the polymer comprises a polyester, polycarbonate, polyamide, or polyether, or unit thereof. In other embodiments of any one of the methods or compositions provided, the polymer comprises poly(ethylene glycol) (PEG), polypropylene glycol, poly(lactic acid), poly(glycolic acid), poly(lactic-co-glycolic acid), or a polycaprolactone, or unit thereof. In some embodiments of any one of the methods or compositions provided, it is preferred that the polymer is biodegradable. Therefore, in these embodiments of any one of the methods or compositions provided, it is preferred that if the polymer comprises a polyether, such as poly(ethylene glycol) or polypropylene glycol or unit thereof, the polymer comprises a block-co-polymer of a polyether and a biodegradable polymer such that the polymer is biodegradable. In other embodiments of any one of the methods or compositions provided, the polymer does not solely comprise a polyether or unit thereof, such as poly(ethylene glycol) or polypropylene glycol or unit thereof.

Other examples of polymers suitable for use in the present invention include, but are not limited to polyethylenes, polycarbonates (e.g. poly(1,3-dioxan-2one)), polyanhydrides (e.g. poly(sebacic anhydride)), polypropylfumerates, polyamides (e.g. polycaprolactam), polyacetals, polyethers, polyesters (e.g., polylactide, polyglycolide, polylactide-co-glycolide, polycaprolactone, polyhydroxyacid (e.g. poly(β-hydroxyalkanoate))), poly(orthoesters), polycyanoacrylates, polyvinyl alcohols, polyurethanes, polyphosphazenes, polyacrylates, polymethacrylates, polyureas, polystyrenes, and polyamines, polylysine, polylysine-PEG copolymers, and poly(ethyleneimine), poly(ethylene imine)-PEG copolymers.

In some embodiments of any one of the methods or compositions provided, polymers in accordance with the present invention include polymers which have been approved for use in humans by the U.S. Food and Drug Administration (FDA) under 21 C.F.R. § 177.2600, including but not limited to polyesters (e.g., polylactic acid, poly(lactic-co-glycolic acid), polycaprolactone, polyvalerolactone, poly(1,3-dioxan-2one)); polyanhydrides (e.g., poly(sebacic anhydride)); polyethers (e.g., polyethylene glycol); polyurethanes; polymethacrylates; polyacrylates; and polycyanoacrylates.

In some embodiments of any one of the methods or compositions provided, polymers can be hydrophilic. For example, polymers may comprise anionic groups (e.g., phosphate group, sulphate group, carboxylate group); cationic groups (e.g., quaternary amine group); or polar groups (e.g., hydroxyl group, thiol group, amine group). In some embodiments of any one of the methods or compositions provided, a synthetic nanocarrier comprising a hydrophilic polymeric matrix generates a hydrophilic environment within the synthetic nanocarrier. In some embodiments of any one of the methods or compositions provided, polymers can be hydrophobic. In some embodiments of any one of the methods or compositions provided, a synthetic nanocarrier comprising a hydrophobic polymeric matrix generates a hydrophobic environment within the synthetic nanocarrier. Selection of the hydrophilicity or hydrophobicity of the polymer may have an impact on the nature of materials that are incorporated within the synthetic nanocarrier.

In some embodiments of any one of the methods or compositions provided, polymers may be modified with one or more moieties and/or functional groups. A variety of moieties or functional groups can be used in accordance with the present invention. In some embodiments of any one of the methods or compositions provided, polymers may be modified with polyethylene glycol (PEG), with a carbohydrate, and/or with acyclic polyacetals derived from polysaccharides (Papisov, 2001, ACS Symposium Series, 786:301). Some embodiments may be made using the general teachings of U.S. Pat. No. 5,543,158 to Gref et al., or WO publication WO2009/051837 by von Andrian et al.

In some embodiments of any one of the methods or compositions provided, polymers may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments of any one of the methods or compositions provided, exemplary polyesters include, for example, polyhydroxyacids; PEG copolymers and copolymers of lactide and glycolide (e.g., PLA-PEG copolymers, PGA-PEG copolymers, PLGA-PEG copolymers, and derivatives thereof. In some embodiments of any one of the methods or compositions provided, polyesters include, for example, poly(caprolactone), poly(caprolactone)-PEG copolymers, poly(L-lactide-co-L-lysine), poly(serine ester), poly(4-hydroxy-L-proline ester), poly[α-(4-aminobutyl)-L-glycolic acid], and derivatives thereof.

In some embodiments of any one of the methods or compositions provided, a polymer may be PLGA. PLGA is a biocompatible and biodegradable co-polymer of lactic acid and glycolic acid, and various forms of PLGA are characterized by the ratio of lactic acid:glycolic acid. Lactic acid can be L-lactic acid, D-lactic acid, or D,L-lactic acid. The degradation rate of PLGA can be adjusted by altering the lactic acid:glycolic acid ratio. In some embodiments of any one of the methods or compositions provided, PLGA to be used in accordance with the present invention is characterized by a lactic acid:glycolic acid ratio of approximately 85:15, approximately 75:25, approximately 60:40, approximately 50:50, approximately 40:60, approximately 25:75, or approximately 15:85.

In some embodiments, polymers can be degradable polyesters bearing cationic side chains (Putnam et al., 1999, Macromolecules, 32:3658; Barrera et al., 1993, J. Am. Chem. Soc., 115:11010; Kwon et al., 1989, Macromolecules, 22:3250; Lim et al., 1999, J. Am. Chem. Soc., 121:5633; and Zhou et al., 1990, Macromolecules, 23:3399). Examples of these polyesters include poly(L-lactide-co-L-lysine) (Barrera et al., 1993, J. Am. Chem. Soc., 115:11010), poly(serine ester) (Zhou et al., 1990, Macromolecules, 23:3399), poly(4-hydroxy-L-proline ester) (Putnam et al., 1999, Macromolecules, 32:3658; and Lim et al., 1999, J. Am. Chem. Soc., 121:5633), and poly(4-hydroxy-L-proline ester) (Putnam et al., 1999, Macromolecules, 32:3658; and Lim et al., 1999, J. Am. Chem. Soc., 121:5633).

The properties of these and other polymers and methods for preparing them are well known in the art (see, for example, U.S. Pat. Nos. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and U.S. Pat. No. 4,946,929; Wang et al., 2001, J. Am. Chem. Soc., 123:9480; Lim et al., 2001, J. Am. Chem. Soc., 123:2460; Langer, 2000, Acc. Chem. Res., 33:94; Langer, 1999, J. Control. Release, 62:7; and Uhrich et al., 1999, Chem. Rev., 99:3181). More generally, a variety of methods for synthesizing certain suitable polymers are described in Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al., Prentice-Hall, 1981; Deming et al., 1997, Nature, 390:386; and in U.S. Pat. Nos. 6,506,577, 6,632,922, 6,686,446, and 6,818,732.

In some embodiments of any one of the methods or compositions provided, polymers can be linear or branched polymers. In some embodiments, polymers can be dendrimers. In some embodiments of any one of the methods or compositions provided, polymers can be substantially cross-linked to one another. In some embodiments of any one of the methods or compositions provided, polymers can be substantially free of cross-links. In some embodiments, polymers can be used in accordance with the present invention without undergoing a cross-linking step. It is further to be understood that the synthetic nanocarriers may comprise block copolymers, graft copolymers, blends, mixtures, and/or adducts of any of the foregoing and other polymers. Those skilled in the art will recognize that the polymers listed herein represent an exemplary, not comprehensive, list of polymers that can be of use in accordance with the present invention.

In some embodiments, synthetic nanocarriers do not comprise a polymeric component. In some embodiments, synthetic nanocarriers may comprise metal particles, quantum dots, ceramic particles, etc. In some embodiments, a non-polymeric synthetic nanocarrier is an aggregate of non-polymeric components, such as an aggregate of metal atoms (e.g., gold atoms).

Any immunosuppressant as provided herein can be, in some embodiments of any one of the methods or compositions provided, coupled to synthetic nanocarriers. Immunosuppressants include, but are not limited to, statins; mTOR inhibitors, such as rapamycin or a rapamycin analog (rapalog); TGF-β signaling agents; TGF-β receptor agonists; histone deacetylase (HDAC) inhibitors; corticosteroids; inhibitors of mitochondrial function, such as rotenone; P38 inhibitors; NF-κβ inhibitors; adenosine receptor agonists; prostaglandin E2 agonists; phosphodiesterase inhibitors, such as phosphodiesterase 4 inhibitor; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors and oxidized ATPs. Immunosuppressants also include IDO, vitamin D3, cyclosporine A, aryl hydrocarbon receptor inhibitors, resveratrol, azathiopurine, 6-mercaptopurine, aspirin, niflumic acid, estriol, tripolide, interleukins (e.g., IL-1, IL-10), cyclosporine A, siRNAs targeting cytokines or cytokine receptors and the like.

Examples of mTOR inhibitors include rapamycin and analogs thereof (e.g., CCL-779, RAD001, AP23573, C20-methallylrapamycin (C20-Marap), C16-(S)-butylsulfonamidorapamycin (C16-BSrap), C16-(S)-3-methylindolerapamycin (C16-iRap) (Bayle et al. Chemistry & Biology 2006, 13:99-107)), AZD8055, BEZ235 (NVP-BEZ235), chrysophanic acid (chrysophanol), deforolimus (MK-8669), everolimus (RAD0001), KU-0063794, PI-103, PP242, temsirolimus, and WYE-354 (available from Selleck, Houston, Tex., USA).

In regard to synthetic nanocarriers coupled to immunosuppressants, methods for coupling components to synthetic nanocarriers may be useful. Elements of the synthetic nanocarriers may be coupled to the overall synthetic nanocarrier, e.g., by one or more covalent bonds, or may be attached by means of one or more linkers. Additional methods of functionalizing synthetic nanocarriers may be adapted from Published US Patent Application 2006/0002852 to Saltzman et al., Published US Patent Application 2009/0028910 to DeSimone et al., or Published International Patent Application WO/2008/127532 A1 to Murthy et al.

In some embodiments, the coupling can be a covalent linker. In embodiments, immunosuppressants according to the invention can be covalently coupled to the external surface via a 1,2,3-triazole linker formed by the 1,3-dipolar cycloaddition reaction of azido groups with immunosuppressant containing an alkyne group or by the 1,3-dipolar cycloaddition reaction of alkynes with immunosuppressants containing an azido group. Such cycloaddition reactions are preferably performed in the presence of a Cu(I) catalyst along with a suitable Cu(I)-ligand and a reducing agent to reduce Cu(II) compound to catalytic active Cu(I) compound. This Cu(I)-catalyzed azide-alkyne cycloaddition (CuAAC) can also be referred as the click reaction.

Additionally, covalent coupling may comprise a covalent linker that comprises an amide linker, a disulfide linker, a thioether linker, a hydrazone linker, a hydrazide linker, an imine or oxime linker, an urea or thiourea linker, an amidine linker, an amine linker, and a sulfonamide linker.

Alternatively or additionally, synthetic nanocarriers can be coupled to components directly or indirectly via non-covalent interactions. In non-covalent embodiments, the non-covalent attaching is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. Such couplings may be arranged to be on an external surface or an internal surface of a synthetic nanocarrier. In embodiments of any one of the methods or compositions provided, encapsulation and/or absorption is a form of coupling.

For detailed descriptions of available conjugation methods, see Hermanson G T “Bioconjugate Techniques”, 2nd Edition Published by Academic Press, Inc., 2008. In addition to covalent attachment the component can be coupled by adsorption to a pre-formed synthetic nanocarrier or it can be coupled by encapsulation during the formation of the synthetic nanocarrier.

Synthetic nanocarriers may be prepared using a wide variety of methods known in the art. For example, synthetic nanocarriers can be formed by methods such as nanoprecipitation, flow focusing using fluidic channels, spray drying, single and double emulsion solvent evaporation, solvent extraction, phase separation, milling, microemulsion procedures, microfabrication, nanofabrication, sacrificial layers, simple and complex coacervation, and other methods well known to those of ordinary skill in the art. Alternatively or additionally, aqueous and organic solvent syntheses for monodisperse semiconductor, conductive, magnetic, organic, and other nanomaterials have been described (Pellegrino et al., 2005, Small, 1:48; Murray et al., 2000, Ann. Rev. Mat. Sci., 30:545; and Trindade et al., 2001, Chem. Mat., 13:3843). Additional methods have been described in the literature (see, e.g., Doubrow, Ed., “Microcapsules and Nanoparticles in Medicine and Pharmacy,” CRC Press, Boca Raton, 1992; Mathiowitz et al., 1987, J. Control. Release, 5:13; Mathiowitz et al., 1987, Reactive Polymers, 6:275; and Mathiowitz et al., 1988, J. Appl. Polymer Sci., 35:755; U.S. Pat. Nos. 5,578,325 and 6,007,845; P. Paolicelli et al., “Surface-modified PLGA-based Nanoparticles that can Efficiently Associate and Deliver Virus-like Particles” Nanomedicine. 5(6):843-853 (2010)).

Materials may be encapsulated into synthetic nanocarriers as desirable using a variety of methods including but not limited to C. Astete et al., “Synthesis and characterization of PLGA nanoparticles” J. Biomater. Sci. Polymer Edn, Vol. 17, No. 3, pp. 247-289 (2006); K. Avgoustakis “Pegylated Poly(Lactide) and Poly(Lactide-Co-Glycolide) Nanoparticles: Preparation, Properties and Possible Applications in Drug Delivery” Current Drug Delivery 1:321-333 (2004); C. Reis et al., “Nanoencapsulation I. Methods for preparation of drug-loaded polymeric nanoparticles” Nanomedicine 2:8-21 (2006); P. Paolicelli et al., “Surface-modified PLGA-based Nanoparticles that can Efficiently Associate and Deliver Virus-like Particles” Nanomedicine. 5(6):843-853 (2010). Other methods suitable for encapsulating materials into synthetic nanocarriers may be used, including without limitation methods disclosed in U.S. Pat. No. 6,632,671 to Unger issued Oct. 14, 2003.

In some embodiments, synthetic nanocarriers are prepared by a nanoprecipitation process or spray drying. Conditions used in preparing synthetic nanocarriers may be altered to yield particles of a desired size or property (e.g., hydrophobicity, hydrophilicity, external morphology, “stickiness,” shape, etc.). The method of preparing the synthetic nanocarriers and the conditions (e.g., solvent, temperature, concentration, air flow rate, etc.) used may depend on the materials to be coupled to the synthetic nanocarriers and/or the composition of the polymer matrix.

If synthetic nanocarriers prepared by any of the above methods have a size range outside of the desired range, synthetic nanocarriers can be sized, for example, using a sieve.

Compositions provided herein may comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol).

Compositions according to the invention can comprise pharmaceutically acceptable excipients, such as preservatives, buffers, saline, or phosphate buffered saline. The compositions may be made using conventional pharmaceutical manufacturing and compounding techniques to arrive at useful dosage forms. In an embodiment of any one of the methods or compositions provided, compositions are suspended in sterile saline solution for injection together with a preservative. Techniques suitable for use in practicing the present invention may be found in Handbook of Industrial Mixing: Science and Practice, Edited by Edward L. Paul, Victor A. Atiemo-Obeng, and Suzanne M. Kresta, 2004 John Wiley & Sons, Inc.; and Pharmaceutics: The Science of Dosage Form Design, 2nd Ed. Edited by M. E. Auten, 2001, Churchill Livingstone. In an embodiment of any one of the methods or compositions provided, compositions are suspended in sterile saline solution for injection with a preservative.

It is to be understood that the compositions of the invention can be made in any suitable manner, and the invention is in no way limited to compositions that can be produced using the methods described herein. Selection of an appropriate method of manufacture may require attention to the properties of the particular moieties being associated.

In some embodiments of any one of the methods or compositions provided, compositions are manufactured under sterile conditions or are terminally sterilized. This can ensure that resulting compositions are sterile and non-infectious, thus improving safety when compared to non-sterile compositions. This provides a valuable safety measure, especially when subjects receiving the compositions have immune defects, are suffering from infection, and/or are susceptible to infection.

Administration according to the present invention may be by a variety of routes, including but not limited to subcutaneous, intravenous, and intraperitoneal routes. The compositions referred to herein may be manufactured and prepared for administration using conventional methods.

The compositions of the invention can be administered in effective amounts, such as the effective amounts described herein. In some embodiments of any one of the methods or compositions provided, repeated multiple cycles of administration of rITs with or without administration of synthetic nanocarriers comprising an immunosuppressant is undertaken. Doses of dosage forms may contain varying amounts of immunosuppressants and/or rITs, according to the invention. The amount of immunosuppressants and/or rITs present in the dosage forms can be varied according to the nature of the rIT, synthetic nanocarrier and/or immunosuppressant, the therapeutic benefit to be accomplished, and other such parameters. In embodiments, dose ranging studies can be conducted to establish optimal therapeutic amounts of the component(s) to be present in dosage forms. In embodiments, the component(s) are present in dosage forms in an amount effective to generate a tolerogenic immune response to the rIT. In preferable embodiments, the component(s) are present in dosage forms in an amount effective reduce immune responses to the rIT, such as when concomitantly administered to a subject. It may be possible to determine amounts of the component(s) effective to generate desired or reduce undesired immune responses using conventional dose ranging studies and techniques in subjects. Dosage forms may be administered at a variety of frequencies.

Aspects of the invention relate to determining a protocol for the methods of administration as provided herein. A protocol can be determined by varying at least the frequency, dosage amount of the rITs and/or synthetic nanocarriers comprising an immunosuppressant and subsequently assessing a desired or undesired immune response. A preferred protocol for practice of the invention reduces an immune response against the rITs and/or allows for repeated administrations as compared to the same method of administrations without administration with synthetic nanocarriers comprising an immunosuppressant as provided herein. The protocol can comprise at least the frequency of the administration and doses of the rITs and/or synthetic nanocarriers comprising an immunosuppressant. Any one of the methods provided herein can include a step of determining a protocol or the administering steps are performed according to a protocol that was determined to achieve any one or more of the desired results as provided herein.

The compositions and methods described herein can be used for subject having or at risk of having conditions such as cancer. Examples of cancer include, but are not limited to breast cancer; biliary tract cancer; bladder cancer; brain cancer including glioblastomas and medulloblastomas; cervical cancer; choriocarcinoma; colon cancer; endometrial cancer; esophageal cancer; gastric cancer; hematological neoplasms including acute lymphocytic and myelogenous leukemia, e.g., B Cell CLL; T-cell acute lymphoblastic leukemia/lymphoma; hairy cell leukemia; chronic myelogenous leukemia, multiple myeloma; AIDS-associated leukemias and adult T-cell leukemia/lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor.

Another aspect of the disclosure relates to kits. In some embodiments, the kit comprises any one or more of the compositions provided herein. In some embodiments, the kit comprises an immunosuppressant, synthetic nanocarrier and rIT. The kit may further comprise a checkpoint inhibitor in some embodiments. In one embodiment, the immunosuppressant is coupled to the synthetic nanocarrier. The various components of the kit can each be contained within separate containers in the kit. In some embodiments, the container is a vial or an ampoule. In some embodiments, the components of the kit are contained within a solution separate from the container, such that the components may be added to the container at a subsequent time. In some embodiments, the components of the kit are in lyophilized form in a separate container, such that they may be reconstituted at a subsequent time. In some embodiments, the kit further comprises instructions for coupling, reconstitution, mixing, administration, etc. In some embodiments, the instructions include a description of the methods described herein. Instructions can be in any suitable form, e.g., as a printed insert or a label. In some embodiments, the kit further comprises one or more syringes or other means for administering the synthetic nanocarrier and rIT and/or checkpoint inhibitor. Preferably, the composition(s) is/are in an amount to provide any one or more doses as provided herein.

### EXAMPLES

### Example 1: Synthesis of Synthetic Nanocarriers Comprising an Immunosuppressant (Prophetic)

Synthetic nanocarriers comprising an immunosuppressant, such as rapamycin, can be produced using any method known to those of ordinary skill in the art. Preferably, in some embodiments of any one of the methods or compositions provided herein the synthetic nanocarriers comprising an immunosuppressant are produced by any one of the methods of US Publication No. US 2016/0128986 A1 and US Publication No. US 2016/0128987 A1, the described methods of such production and the resulting synthetic nanocarriers being incorporated herein by reference in their entirety. In any one of the methods or compositions provided herein, the synthetic nanocarriers comprising an immunosuppressant are such incorporated synthetic nanocarriers.

### Example 2: Concomitant Administration of a Recombinant Immunotoxin with Synthetic Nanocarriers Coupled to Immunosuppressant (Prophetic)

A rIT is administered concomitantly, such as on the same day, as a synthetic nanocarrier composition of any one of the Examples to subjects recruited for a clinical trial. One or more immune responses against the rIT is evaluated. The level(s) of the one or more immune responses against the rIT can be evaluated by comparison with the level(s) of the one or more immune responses in the subjects, or another group of subjects, administered the rIT in the absence of the synthetic nanocarrier composition, such as when administered the rIT alone. In embodiments, any protocol of administration is evaluated in a similar manner.

In an application of the information established during such trials, the rIT and synthetic nanocarrier composition can be administered concomitantly to subjects in need of rIT therapy when such subjects are expected to have an undesired immune response against the rIT when not administered concomitantly with the synthetic nanocarrier composition. In a further embodiment, a protocol using the information established during the trials can be prepared to guide the concomitant dosing of the rIT and synthetic nanocarriers of subjects in need of treatment with a rIT and have or are expected to have an undesired immune response against the rIT without the benefit of the synthetic nanocarrier composition. The protocol so prepared can then be used to treat subjects, particularly human subjects.

### Example 3: Tolerogenic Synthetic Nanocarriers Restore the Anti-Tumor Activity of Recombinant Immunotoxins by Mitigating Immunogenicity

The immune response rITs is a major factor limiting their efficacy against, for example, solid tumors in cancer patients with intact immune systems. Here, antigen-specific immune tolerance for rITs using rapamycin encapsulated in synthetic nanocarriers (SVP-R) was studied. These nanocarriers are comprised of a biodegradable poly (lactic acid) core with a corona of surface PEGylation. It was demonstrated that SVP-R produce a long lasting, specific and transferable immune tolerance that prevents ADA formation against LMB-100 in naïve mice and reduces ADAs in mice with pre-existing antibodies to the rIT. Induction of immune tolerance to LMB-100 resulted in restoration of its anti-tumor activity in a syngeneic mesothelioma tumor model in an immunocompetent mouse that would otherwise be neutralized by ADAs.

Combination of LMB-100 with SVP-R Prevents ADA Response

To evaluate the effect of synthetic nanocarriers comprising rapamycin on the ADA response to LMB-100 (FIG. 2A), BALB/c mice were injected every other week with LMB-100, or a combination of LMB-100+SVP-R. LMB-100 has mutations that diminish human but not mouse responses. Mice injected with LMB-100 had a strong and rapid response to LMB-100 (FIG. 2B) with a mean titer of 10,975±2372 at week 14, indicating that LMB-100 is immunogenic in BALB/c mice.

All mice injected with LMB-100+SVP-R had an undetectable titer during the entire course of the experiment, indicating effective prevention of ADA formation. Furthermore, mice injected seven times with LMB-100 and given SVP-R with only the first three injections had a mean titer of only 371±301 at week 14, indicating induction of immune tolerance. This titer was significantly lower than the titer of control mice treated with LMB-100 alone at both week 8 (p=0.03), after only four doses, and at week 14 (p=0.0006), after seven doses. The area under the curve (AUC) for each mouse throughout the experiment was calculated to compare the ADA responses (FIG. 3A) and demonstrated a significant decrease in mice given three doses (p=0.001) or seven doses of SVP-R (p=0002). The mice tolerated treatment well, with no significant observed weight loss (FIG. 3B).

### Timing of SVP-R Immunization is Important for Immune Tolerance

To determine the efficacy of SVP-R with an LMB-100 regimen similar to that used in patients, mice were treated with successive cycles of LMB-100. Each cycle consisted of three doses per week (QODx3) every other week, and mice were injected with SVP-R once, twice or three times during the first and second cycles (FIG. 2C). It was found that a single dose of SVP-R per cycle was as effective as three doses in preventing ADA formation (p=0.003). The median titers in mice receiving LMB-100 alone were 47,926, compared to only 881, 1958 and 993 in mice immunized with LMB-100+SVP-R given 2, 4, or 6 times, respectively, over the two treatment cycles. The ADA suppression was also maintained when mice were challenged with three additional cycles of LMB-100 in the absence of further SVP-R treatment. Six doses of LMB-100+SVP-R were well tolerated by the mice, with no significant weight loss (FIG. 3C).

The effect of timing of SVP-R treatment was evaluated by staggering the day of SVP-R injection. LMB-100 was injected on days 1, 3, and 5 of each of five cycles, and co-administered SVP-R on day 1, day 3, days 1+3, days 3+5 or days 1+3+5 of each cycle (FIG. 2D). Control mice treated with LMB-100 showed a mean titer of 44,132 at the end of five treatment cycles. In contrast, mice that received SVP-R on day 1, regardless of whether they received one, two or three SVP-R doses during each cycle, showed significant decreases in ADA formation, with mean titers of 1,413±495 (p=0.0007), 2,952±1,320 (p=0.001) and 1,979±807 (p=0.0007), respectively. Mice that received SVP-R on day 3 or days 3+5 had final titers of 29,341±11,705 and 41,934±9,725, respectively, indicating that co-treatment with SVP-R on the first day of each cycle is important to prevent ADA formation.

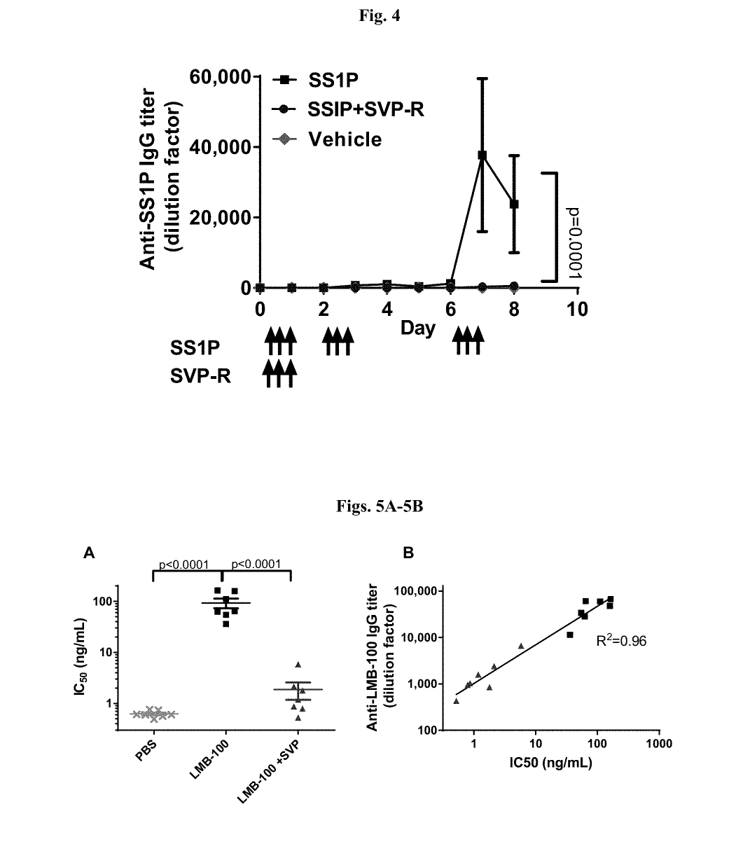

SVP-R was also evaluated with the more immunogenic precursor of LMB-100, SS1P. Mice were injected with three doses of SS1P on days 1, 3 and 7 (FIG. 4), and SVP-R was given on day 1. Three cycles of SS1P induced a mean ADA titer of 37,734±21,748, and a single cycle of SVP-R completely block these ADAs (p=0.0001).

### ADA Response is Neutralizing and Targets Both the Fab and Toxin

To detect if ADAs can neutralize the immunotoxin, a functional in vitro neutralization assay was performed using plasma samples from mice injected with either LMB-100 (15 doses), LMB-100 (15 doses) with SVP-R (6 doses) or vehicle. Plasma samples were mixed with various concentrations of LMB-100 and added to KLM-1 human pancreatic cells. The cells were very sensitive to LMB-100 with an IC50 of 1.1 ng/ml (FIG. 2E). Plasma from mice immunized with LMB-100 alone inhibited the activity of LMB-100 and shifted the IC50 to 93.2 ng/ml (p<0.0001), indicating that the ADAs are neutralizing. In contrast, incubation of LMB-100 with plasma LMB-100+SVP-R showed an IC50 50-fold lower (p<0.0001) and not significantly different from the IC50 of LMB-100 incubated with plasma from vehicle treated mice (FIG. 5A). A strong correlation between the anti LMB-100 titers and IC50 (R2=0.96) was observed (FIG. 5B).

To determine if the ADAs against LMB-100 target the Fab, the toxin fragment or both, the plasma from mice injected with five doses weekly of LMB-100 alone or in combination with SVP-R (n=8) was assayed on plates coated with LMB-100, a human Fab or with an immunotoxin containing the same domain III of the Exotoxin A (PE24), as found in LMB-100, fused to a mouse Fv (anti-TacFv-PE24). Anti-LMB-100 plasma reacted with both components of the immunotoxin (FIG. 2F). As expected, SVP-R reduced the response to both components.

Combination of LMB-100 with SVP-R Induces a Specific and Transferable Immune Tolerance

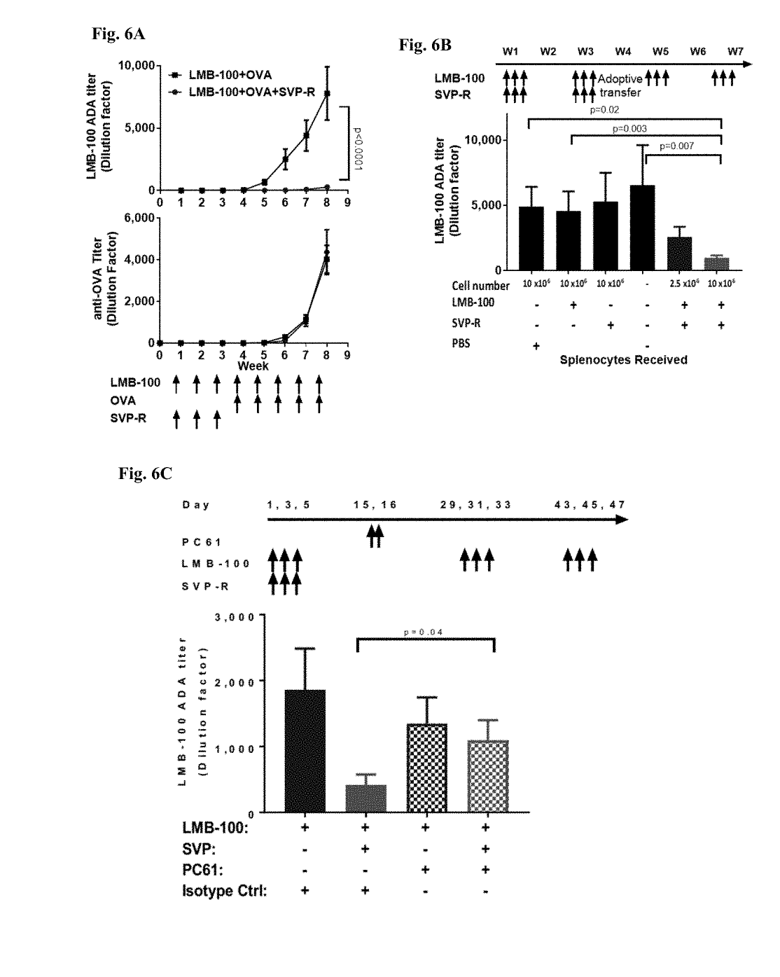

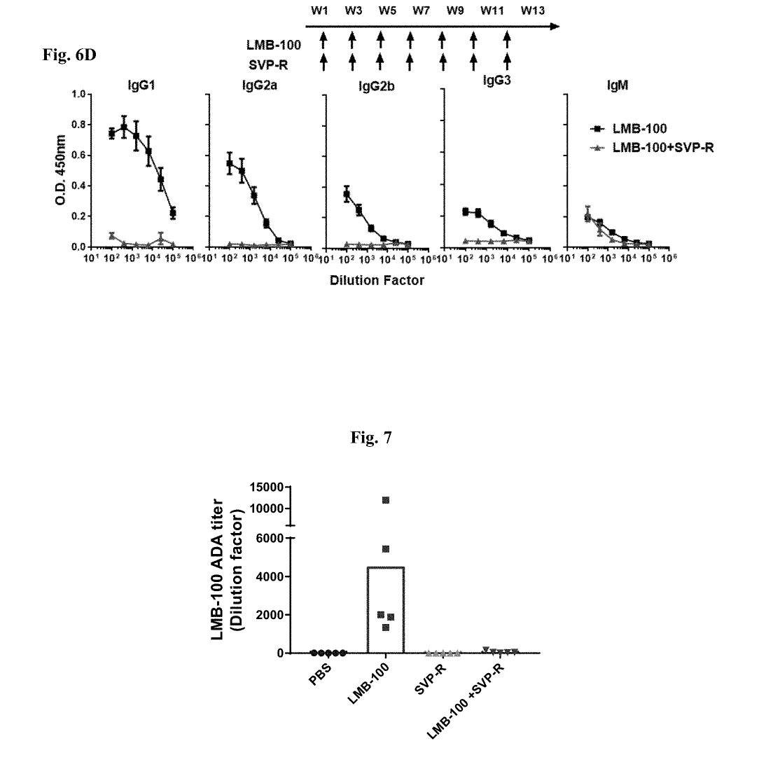

To determine if the suppression of ADA formation is a result of antigen-specific immune tolerance rather than a chronic immune suppression, mice were immunized with eight weekly injections of LMB-100 and three doses of SVP-R (i.v.) at weeks 1, 2, and 3. At week 4, mice were challenged with four weekly injections of ovalbumin and LMB-100 (s.c.) (FIG. 6A). Combination of LMB-100+SVP-R selectively inhibited ADA formation against LMB-100, but did not affect the antibody response to ovalbumin, resulting in similar anti-ovalbumin titers of 4,362 and 4,024. These results indicate that the combination of LMB-100 with SVP-R induced a specific immune tolerance that did not suppress the ability of the mice to mount an immune response against another antigen administered later.

To test whether the immune tolerance could be transferred from tolerant mice to naïve mice, donor mice were treated with either LMB-100, SVP-R or LMB-100+SVP-R for two cycles. Mice immunized with LMB-100 alone showed a mean titer of 4521±1994, compared to 51±25 in mice treated with LMB-100+SVP-R (FIG. 7). Splenocytes were isolated, pooled and transferred to naïve recipient mice (FIG. 6B). One week after cell injection, all recipient mice were challenged with two cycles of LMB-100. Adoptive transfer of cells from mice immunized with LMB-100 followed by LMB-100 challenge of recipient mice induced a mean titer of 4884±1548, which was not significantly different from the titer in mice receiving cells from vehicle-treated mice or no cells (mean titers of 4571±1494 and 6541±3079, respectively). The adoptive transfer did not induce substantial immune memory. Because these three mice groups had similar mean titers, these mice are referred to as controls.

In contrast, adoptive transfer of 10×10⁶ splenocytes from mice immunized with a combination of LMB-100+SVP-R decreased the titers by 78%-85% compared to the controls (p=0.007, 0.003 and 0.02, respectively). Adoptive transfer of 2.5×10⁶ splenocytes reduced titers by 44%-61%, but was not statistically significant (p=0.5). The mean titer of mice that received splenocytes from SVP-R treated mice was not different from the control mice, indicating that tolerance induction required both LMB-100 and SVP-R in the donor mice and was not due to a general immune suppression.

### Depletion of Treg Cells

To study the role of Treg cells in SVP-R-induced immune tolerance, Treg cells were depleted in vivo after SVP-R tolerance induction. Mice were injected with LMB-100 or LMB-100+SVP-R three times. On days 15 and 16 Treg cells were depleted using an anti CD25 (PC61) depleting antibody²³, and were challenged with two more cycles of LMB-100 (FIG. 6C). The depletion of Tregs abrogated the tolerogenic effect of SVP-R, increasing the mean titer from 416±157 to 1094±304 (p=0.04). This titer of 1094 was similar to the titer in mice that did not receive SVP-R (1348±399).

### Ig Subclasses

To study the effect of SVP-R on class switching, plasma samples were characterized for LMB-100-specific IgG and IgM antibodies (FIG. 6D). Immunization with LMB-100 induced ADAs distributed across all IgG subclasses, with IgG1 as the most dominant. This subclass distribution is similar to the IgG subclass distribution previously described after immunization with the parent immunotoxin SS1P²⁴. Immunization with LMB-100+SVP-R induced an undetectable signal of LMB-100-specific IgG1, IgG2a, IgG2b or IgG3 antibodies. Interestingly, the levels of anti-LMB-100 IgM antibodies was similar to the level in mice immunized with LMB-100 alone. These results indicate that SVP-R prevents isotype switching but does not prevent IgM production.

LMB-100 with SVP-R Co-Localizes Preferentially on Dendritic Cells and Macrophages

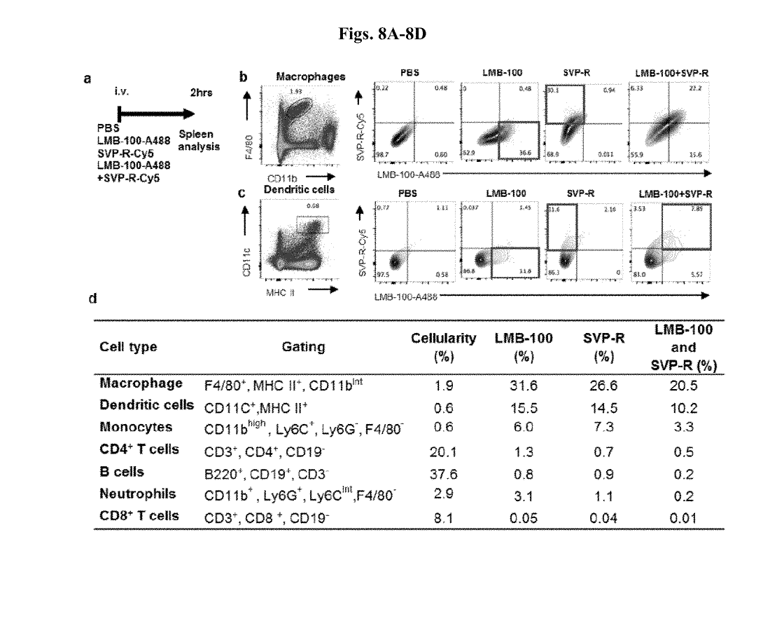

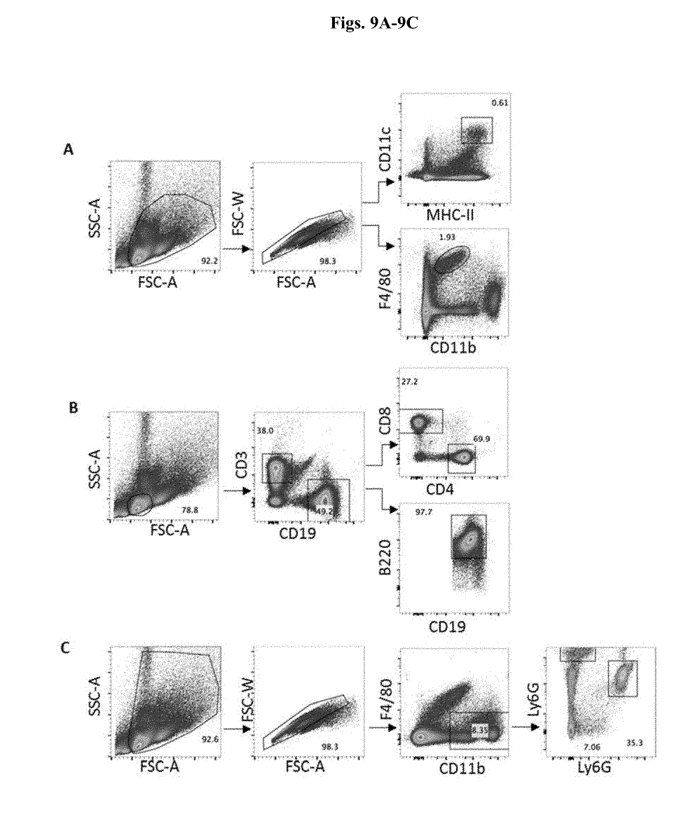

To determine the fate of SVP-R and LMB-100 in the spleen, after injection in vivo, Alexa-488 labeled LMB-100 and Cy5 labeled SVP-R were consecutively injected and the splenocytes were isolated 2 hours post-injection (FIG. 8A). Cell phenotype was analyzed using cellular markers according to the gating strategy shown. The uptake of LMB-100 and SVP-R was compared in macrophages, DC, CD4+ and CD8+ T cells, B cells, neutrophils and monocytes (FIGS. 8B-8D). It was found that macrophages and DC had the highest uptake of both LMB-100 and SVP-R; 38% of the macrophages and 13% of the DC were positive for LMB-100 and 29% of the macrophages and 11% of the DC were positive for SVP-R. Interestingly, 22% of the macrophages and 9% of the DC stained positive for both. This co-localization occurred even though the two agents were injected separately. Relative cell numbers were not changed (Table 1). Monocytic cells that express CD11b^{high} Ly6C+ and Ly6G− have been involved in immune suppressive activity^{25,26}. It was found that 3% of these cells demonstrated uptake of both LMB-100 and SVP-R. Finally, lymphocytes and neutrophils displayed the lowest percentages of co-localization (FIG. 8D). Together these results suggest that SVP-R and LMB-100 preferential uptake by professional antigen presenting cells might mediate the immune tolerance.

TABLE 1
Cellularity in Mice Spleens Two Hours after Injection with LMB-100 and
Synthetic Nanocarriers Comprising Rapamycin

| | | Concentration in mouse spleen (%) | | | |
|---|---|---|---|---|---|
| | | | | | LMB-100- |
| Cell phenotype Gating | | Naïve | LMB-100 | SVP-R | SVP-R |
| Macrophage | CD11C⁺, MHC II⁺ | 2.0 ± 0.3 | 1.7 ± 0.2 | 2.0 ± 0.1 | 1.9 ± 0.0 |
| DC | F4/80⁺, MHC II⁺, CD11b^{Int} | 0.7 ± 0.2 | 0.7 ± 0.1 | 0.6 ± 0.2 | 0.7 ± 0.2 |
| Neutrophils | B220⁺, CD19⁺, CD3⁻ | 2.9 ± 1.1 | 2.2 ± 0.1 | 3.0 ± 0.1 | 2.6 ± 0.0 |
| “Monocytes” | CD3⁺, CD4⁺, CD19⁻ | 0.5 ± 0.1 | 0.5 ± 0.1 | 0.6 ± 0.1 | 0.5 ± 0.1 |
| B cells | CD3⁺, CD8⁺, CD19⁻ | 47.1 ± 5.4 | 51.3 ± 5.4 | 46.1 ± 6.4 | 48.1 ± 5.3 |
| CD4 | CD11b⁺, Ly6G⁺, Ly6C^{Int}, F4/80⁻ | 26.9 ± 4.1 | 23.6 ± 2.5 | 27.1 ± 4.3 | 25.9 ± 3.6 |
| CD8 | CD11b^{high}, Ly6C⁺, Ly6G⁻, F4/80⁻ | 10.7 ± 1.5 | 9.4 ± 1.3 | 11.0 ± 2.7 | 10.5 ± 3.4 |

Combination of LMB-100 with SVP-R Prevents ADA Response in Mice with Pre-Existing Antibodies

To determine if SVP-R could reduce immunogenicity and induce immune tolerance in mice with pre-existing ADAs to the rIT, mice were immunized six times with LMB-100 during weeks 1 and 3 to induce pre-existing ADAs. At week 9, mice had a mean titer of 741±66 and were divided into three groups with similar mean titers. At week 10, the groups were immunized with vehicle (PBS), LMB-100 or LMB-100+SVP-R. Titers were evaluated at week 12. Challenge with LMB-100 alone induced a strong memory immune response resulting in a mean ADA titer of 9808±3608. In contrast, challenge with LMB-100+SVP-R not only prevented the antibody increase but decreased the titer (titer=257±121) compared to the pre-boost titer (738±320, p=0.003) and compared to mice that were injected with PBS at week 12 (titer=502±143, p=0.002). This response was observed in three additional experiments with groups of 8, 8 and 4 mice.

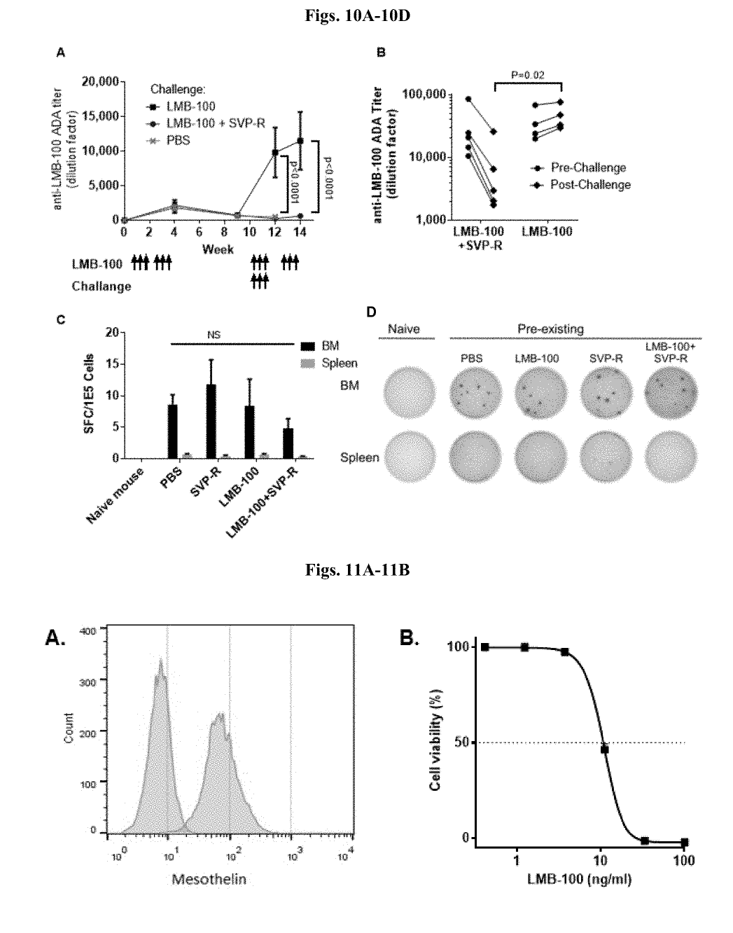

To evaluate if SVP-R can induce a lasting immune tolerance that can prevent a response to later challenges in such mice, the mice were challenged with three additional doses of LMB-100 (no SVP-R) on week 13 (FIG. 10A). Titer evaluation at week 14 showed that administration of LMB-100+SVP-R on week 10 maintained a low titer of 634±269 which was significantly lower than the titer of mice treated with LMB-100 alone (11505±4172, p=0.0001). This indicates that the SVP-R+LMB-100 combination on week 10 induced an immune tolerance which prevented the response to later LMB-100 challenge.

Next, whether SVP-R could also be used to reduce high titers of pre-existing antibodies to the rIT was evaluated. Control mice from FIG. 10A which had anti-LMB-100 antibody titers >10,000 induced by 12 doses of LMB-100 over the course of 14 weeks were injected with LMB-100 or LMB-100+SVP-R (FIG. 10B). Mice treated with the combination had a significant decrease in titer from 31,114±13,730 to 7,797±4,558 (p=0.02).

To determine if treatment of mice with pre-existing antibodies with the combination affected the number of antibody secreting plasma cells in the bone marrow (BM), mice were treated with pre-existing antibodies to the rIT with either PBS, LMB-100, SVP-R or a combination of two. Cells collected from BM and spleen 24 hours after injection and were assayed for the number of cells making anti-LMB-100 antibodies by ELISpot (FIGS. 10C-10D). All mice had a similar number of antibody secreting cells (mean=9.6±6.7 SFC/1E6 cells) in the BM and no detectable spots in their spleens. These results indicate that SVP-R does not affect antibody secreting plasma cells residing in the BM.

Combination of LMB-100 with SVP-R Restores Anti-Tumor Activity of LMB-100 in Mice with Pre-Existing Anti-LMB-100 Antibodies

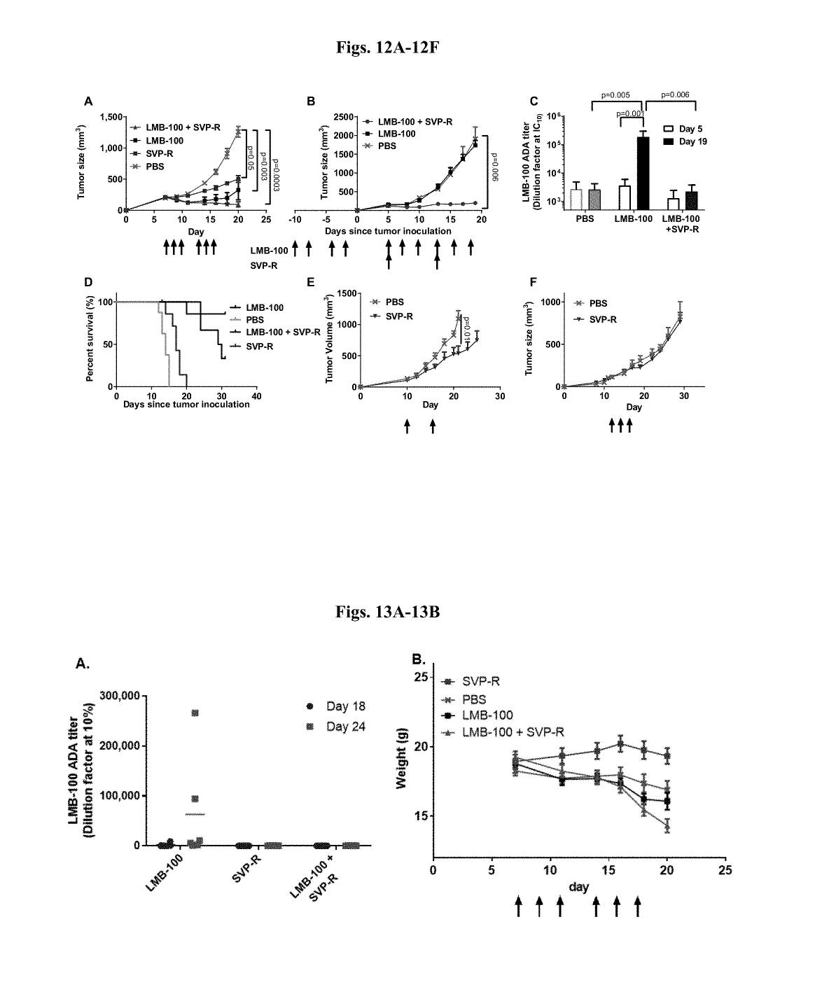

To study the activity of LMB-100 and SVP-R in immunocompetent tumor bearing mice, the AB-1 mouse mesothelioma cell line²⁷ was stably transfected with human mesothelin (AB1-L9, FIG. 11A-11B). AB1-L9 cells inoculated into BALB/c mice grew rapidly, reaching a size of 600 mm³ in 15 days (FIG. 12A). To evaluate anti-tumor activity, tumor-bearing mice were therapeutically treated six times with LMB-100, SVP-R or a combination of the two, when the tumors reached a mean size of 199 mm³. Mice treated with LMB-100 (black line) showed significant tumor growth inhibition (p=0.003 for AUC of tumor growth curves compared to PBS treated mice) with 1/7 mice achieving complete remission. Mice that were treated with SVP-R showed only a minor tumor growth delay (p=0.05). However, LMB-100+SVP-R induced the most significant tumor growth inhibition (p=0.0003) resulting in a 13-fold decrease in tumor size on day 20. Due to the relatively short immunization schedule, all mice had either very low or undetectable titers when evaluated on day 18 of the experiment (FIG. 13A), so no significant in vivo neutralization of LMB-100 was observed.

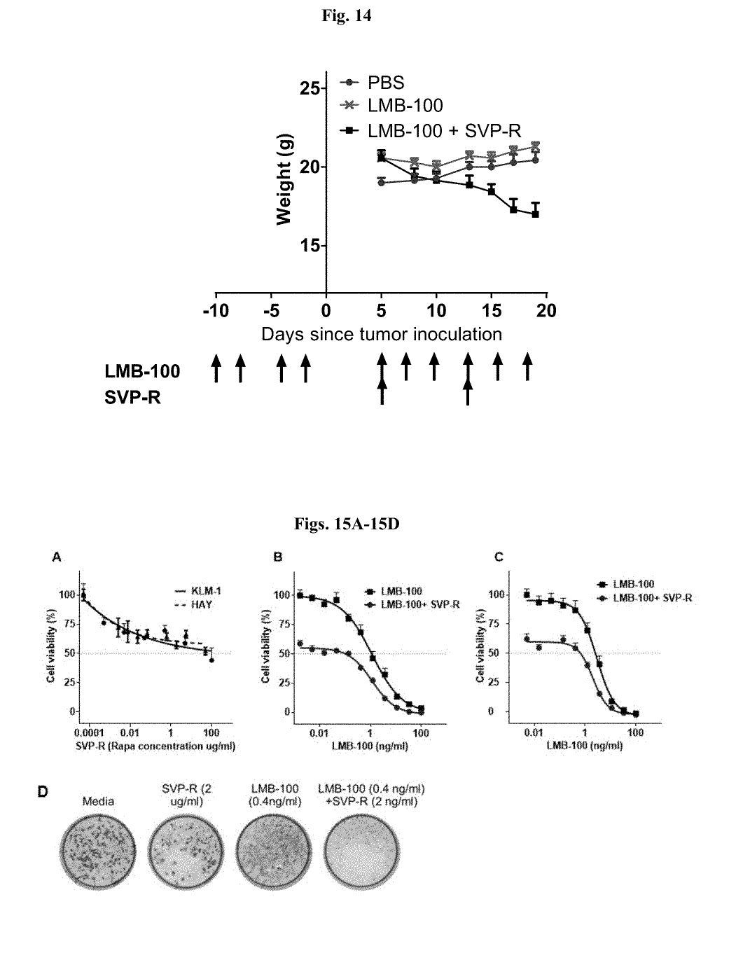

To study the activity of LMB-100 and SVP-R in mice with pre-existing antibodies to the rIT, mice were first immunized with LMB-100 four times to induce an average baseline titer of 2597±2080 prior to inoculation with AB1-L9. Five days after tumor inoculation, when the tumors reached a mean of 135 mm³, mice were treated with two cycles of three injections with LMB-100 or vehicle (FIG. 12B) with or without SVP-R administered on the first day of each cycle (every other week). It was found that the tumors treated with LMB-100 alone did not respond to treatment, and had a similar growth rate as PBS-treated tumors. The lack of response to LMB-100 was attributed to the high ADA titer (FIG. 12C) that neutralized the activity of LMB-100. In contrast, mice treated with the SVP-R+LMB-100 had an excellent response to LMB-100 and did not develop high ADA titers. Mice treated LMB-100+SVP-R had a higher survival rate (time to reach 600 mm³) (p=0.0001) (FIG. 12D). These experiments were repeated two more times using seven mice per group with similar results. However, mice treated with LMB-100+SVP-R showed decreased weight, perhaps due to increased exposure to LMB-100 as a result of preventing neutralizing ADAs (FIG. 14).

SVP-R does not Accelerate Tumor Growth Rate

To test if treating mice with SVP-R interferes with tumor immunity and/or enhances tumor growth, the CT26 (murine colon carcinoma) and 66C14 (murine breast cancer) cell line was inoculated in the flank of immune competent BALB/c mice and the growth rate in SVP-R treated mice was compared to that of the PBS treated mice (FIGS. 12E-12F). SVP-R delayed tumor CT-26 tumor growth and showed no change in tumor growth in 66C14 tumors.

### SVP-R Enhances the Cytotoxic Activity of LMB-100 in Human Cell Lines

Because rapamycin has also been reported to have anti-tumor activity, the cytotoxic activity of the combination on human mesothelioma cells (HAY) and human pancreatic cells (KLM-1) in vitro was measured. It was found that SVP-R had modest cytotoxic activity by itself (FIG. 15A) in both cell lines. However, when combined with LMB-100, 5 μg/ml of SVP-R improved the cytotoxic activity of LMB-100, shifting the IC50 on KLM-1 cells from 1.1 ng/ml to 0.1 ng/ml (FIG. 15B) and on HAY cells 1 μg/ml of SVP-R improved the IC50 from 2.9 ng/ml to 0.9 ng/ml (FIG. 15C). HAY cell viability was also evaluated by staining with crystal violet after a 72 hour incubation with SVP-R (2m/ml) and LMB-100 (0.4 ng/ml) followed by incubation for 72 hours with no drug (FIG. 15D). The combination was more effective than either drug alone in killing cells.

### SVP-R Activity is not Diminished by Checkpoint Inhibitors or Co-Stimulatory Agonists

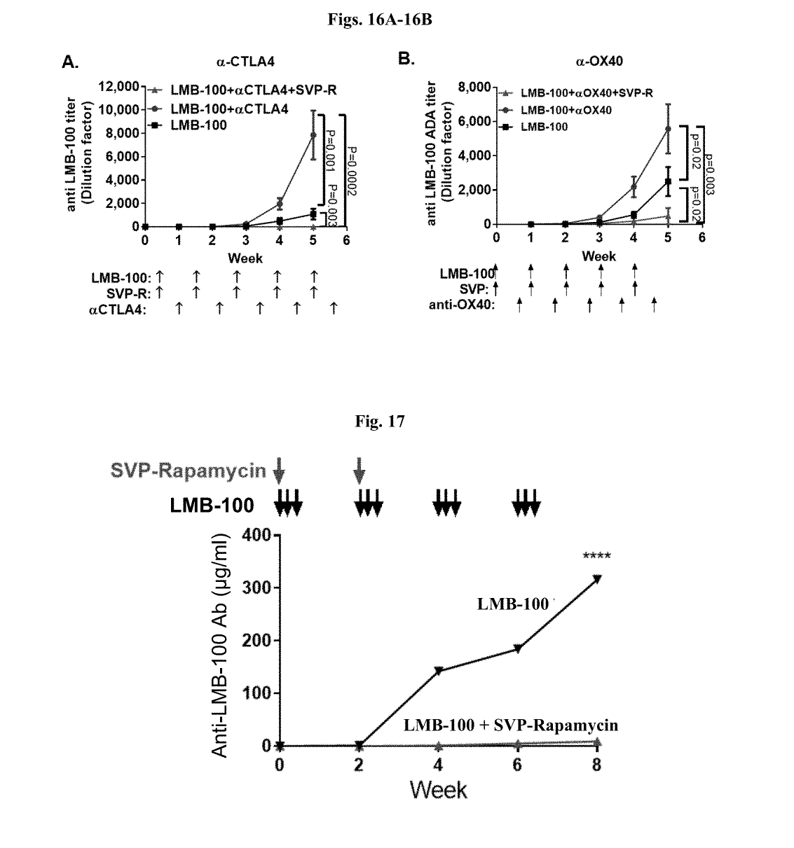

Whether anti-CTLA-4 antagonist antibody and anti-OX-40 agonist antibody can enhance the formation of ADAs against LMB-100 was investigated, as well as whether such ADAs could be blocked by SVP-R. Mice were injected with five weekly doses of LMB-100 and an anti-mouse CTLA-4 antibody or an anti-OX-40 antibody given on the fifth day of every week (FIGS. 16A-16B), n=8. It was found that both antibodies substantially enhanced the formation of anti-LMB-100 ADA titers compared to treatment with LMB-100 alone (p=0.001 and p=0.02 for anti-CTLA-4 and anti-OX-40, respectively). Injection of SVP-R on the same days as LMB-100 resulted in either elimination (mean titer was below the limit of detection) or a dramatic 12-fold decrease in titer in the mice treated with anti-CTLA-4 or anti-OX-40, respectively. SVP-R activity was not compromised by the activity of the immune checkpoint inhibitors or co-stimulatory agonists. These experiments were repeated two more times with n=5 and n=3 with similar results.

### Immune Suppression Versus Tolerance

Previous studies have evaluated several immune suppression approaches to reduce the immunogenicity of rITs in patients. These approaches include B cell depletion using Rituximab, which was ineffective in preventing anti-immunotoxin immune response in patients²⁸ or B and T cell suppression using a combination of cyclophosphamide and pentostatin¹⁴. The success of this approach was limited by the toxicity of the immunosuppressive agents, and while some of the patients had a delay in ADA formation, most patients developed strong ADA responses that halted treatment.

### Immune Tolerance Mechanism

In this study, it is demonstrated that SVP-R specifically targets professional phagocytes such as macrophages and DC and to a lesser extent monocytes. This is unlike general immune suppressive therapies. LMB-100 was found to specifically target professional phagocytes and to co-localize with SVP-R (FIG. 8). The tolerance was abrogated after depletion of Tregs (FIG. 6C), supporting the mechanism of myeloid cell tolerance mediated by Treg cells. In addition, while SVP-R effectively inhibited IgG antibody responses, it was observed that specific IgM antibodies were not inhibited by SVP-R (FIG. 6D). This is also supportive of a Treg-mediated mechanism.

A major differentiator between immune suppression and tolerance is the ability to mount an immune response against other antigens. It was found that mice that were tolerized by injections of LMB-100 and SVP-R mounted an immune response to a second antigen that was injected subcutaneously (FIG. 6A). The fact that the mice had an immune response to the second immunogen but not to LMB-100, even though both were administered at the same time, dose, and frequency during the challenge phase, indicates the induction of specific tolerance to LMB-100 rather than global suppression of the immune system. Immune suppression is commonly mediated by drugs which impart no lasting effect on the immune system after the cessation of therapy. Immune tolerance on the other hand, involves the induction of regulatory cells which actively maintain tolerance in the absence of drugs. It was found that transfer of splenocytes isolated from mice treated with the combination of LMB-100 and SVP-R (FIG. 6B) prevented ADA formation in naïve recipient mice. Together, the data suggest that the combination of LMB-100 with SVP-R induces immune tolerance.

### Activity in a Pre-Existing Antibody Model

The present findings indicate that SVP-R were not only effective in controlling the boost in anti-LMB-100 titers, but actually demonstrated a striking prolonged tolerance (FIG. 10A-10D) with a combination of rITs with SVP-R. Thus, the methods and compositions provided herein may be useful in patients with pre-existing antibodies to rITs (perhaps even in patients that participated in previous clinical trials with SS1P, LMB-100 or Moxetumomab Pasudotox). Many patients in these trials initially responded to immunotoxin therapy, but the response was halted due to ADA formation^{7,30}.

### Rapamycin and Cancer

The mTOR signaling network contains a number of tumor suppressor genes and proto-oncogenes including PTEN, PIK3 and AKT (reviewed in³²). Here, it was found that SVP-R improved the cytotoxic and anti-tumor activity of the immunotoxin (FIGS. 12A and 15A-15D). The release of rapamycin from the synthetic nanocarriers at the tumor site could synergize with the targeted immunotoxin.

### SVP-R Did not Affect Tumor Immunogenicity

Importantly, SVP-R alone did not cause the tumors in immune competent mice to grow faster (FIGS. 12A, 12E-12F). These observations alleviate a potential safety concern of the SVP-R inducing tolerance against the tumor or making the tumor grow faster.

Triple Combination with Checkpoint Inhibitors

The effect of anti-CTLA-4 and anti-OX-40 antibodies on the onset and intensity of ADA formation against LMB-100, and the ability of SVP-R to prevent these responses were evaluated. It was found that both anti-CTLA-4 checkpoint inhibition and anti-OX40 co-stimulatory agonist expedited and intensified the formation of LMB-100 ADAs (FIGS. 16A-16B). Importantly, SVP-R given at the day of injection of LMB-100 completely eradicated these exacerbated immunogenicity responses. The fact that these immune stimulatory mAbs did not compromise the tolerogenic activity of SVP-R suggests that the tolerogenic signal is not overridden by these immunotherapeutic antibodies in the context of the combination therapy as provided herein.

### Materials and Methods

### LMB-100 and SVP-R

LMB-100 was manufactured as previously described⁴¹. SVP-R were manufactured by as previously described with rapamycin content of 500m/ml¹⁹.

### Animal Experiments

Female BALB/cAnNCr mice (8-14 weeks of age) were used. Mice were injected with antigens and SVP-R intravenously unless described otherwise. Mice were injected per the schedules indicated in each experiment (rIT was injected 5 minutes after the SVP-R) and plasma samples were collected by mandibular bleeding. Mice weight was measured weekly. All mouse studies were performed with age-matched control groups.

For tumor experiments, female BALB/c were inoculated with 1×10⁶ AB1-L9 cells or 1×10⁶CT26 cells (ATCC) in RPMI in the flank, or 0.5×10⁶ 66C14 cells in IMDM media in the mammary pad. Tumor sizes were measured using a caliper every two or three days. Mice were euthanized if they experienced a tumor burden greater than 10% body weight. No animals were excluded from statistical analysis³⁷.

Depletion of Treg cells was performed by intraperitoneal (i.p.) injection of 200 μg of anti-mouse CD25 depleting antibody (clone PC61) or isotype control (clone TNP6A7) (both purchased from BioXcell) as previously described²³.

Anti-CTLA-4 (Roche IgG2A, clone 9D9) was provided, and anti-OX40 (clone OX-86, InVivoPlus, BioXcell) was purchased. Antibodies were diluted in PBS and 5 mg/kg were injected i.p. as in the indicated schedules.

### Development of a Syngeneic Mouse Model

Cells were inoculated subcutaneously (s.c.) into the flank of immunocompetent BALB/c mice. However, only 50% of the tumors grew, possibly due to immune rejection of the human transgene. Once tumor volume reached 200 mm³ in some of the mice, tumors were excised, digested and cloned in 96 wells plates with puromycin for selection. Fifteen single clones were obtained and the clone with the highest GeoMean value (FIG. 13A) was evaluated for growth in mice with >95% implantation success.

Cytotoxic activity of AB1-L9 cells was evaluated by treating the cells with LMB-100 and assessing their viability 72 hours later using WST-8 cell counting kit (FIG. 13B). It was found that LMB-100 kills AB1-L9 with an IC50 of 10.6 ng/ml.

### Cytotoxicity and Neutralization Assay

KLM1 pancreatic cell line was provided (NCI, Bethesda, Md.). HAY mesothelioma cells were provided by the Stehlin Foundation for Cancer Research (Houston, Tex.). Cells were cultured in RPMI media supplemented with 10% FCS, 1% L-Glutamine and 1% Penicillin/Streptomycin. Cells were seeded in 96 well flat bottom plates (5,000 cells/well) for 24 hours. Cells were treated with various concentrations of LMB-100, SVP-R or both in four replicas. Cell viability was assessed 72 hours later using a WST cell viability assay (Dojindo Molecular Technologies Inc,) per manufacturer's instructions. Color change was evaluated at optical density (O.D.) 450 nm. O.D reads were normalized between 0 to 100% viability. One hundred percent viability represents no treatment and 0% represents Staurosporine (Sigma-Aldrich) positive control.

Neutralization assays were performed using KLM1 cells as previously described⁴². Serum samples from 21 mice were diluted 1:50.

### ELISA

Total Ig Anti-LMB-100 and Anti-Ova Antibodies:

Plasma samples were collected into heparinized tubes, spun and frozen until titer evaluation. Total Ig anti-LMB-100 and anti-Ova antibodies were measured by a direct ELISA as previously described⁴².

Isotype Determination of Anti-LMB-100 and Total Ig:

ELISA plates (Thermo Fisher) were coated with 2m/ml of LMB-100 or polyclonal donkey anti-mouse IgG (Jackson Immuno Research Laboratories, Inc.). Plates were blocked and serial dilutions of plasma were incubated for 1 hour. Captured antibodies in the plasma were bound by goat anti-mouse IgG1, IgG2a, IgG2b, IgG3 and IgM isotyping kits at dilutions of 1:3,000, 1:4,000, 1:4,000, 1:3000 and 1:16,000, respectfully (Sigma), and anti-goat IgG (H+L) HRP (1:15,000) (Jackson Immuno Research Laboratories, Inc.) was used for detection.

ADA Against the Fab or the Toxin Fragments:

ELISA plates were coated with 2 μg/mL of the Fab portion of LMB-100, or 2m/mL of RIT that contains a murine scFv that targets an irrelevant epitope (anti-Tac) linked to the deimmunized toxin fragment of LMB-100. ADA determination was performed as described above. The O.D. of the wells was read immediately after adding H2SO4 stop solution at a wavelength 450 nm with subtraction at 650 nm. Titers were calculated based on a four-parameter logistic curve-fit graph and interpolated on the half maximal value of the anti-LMB-100 (IP12)¹⁵ or anti-Ova (clone TOSG1C6 Biolegend) standard curves.

Transfection of Cell Line with Human Mesothelin and Tumor Inoculation

AB-1 mouse mesothelioma cell line (Sigma) was stably transfected with human mesothelin cDNA³⁷ by Lipofectamine LTX/PLUS reagents (Invitrogen) per manufacturer's protocol. The transfected cells were sorted three times for the top 5% high expression cells by FACS. LMB-100/SS1P sensitive single clones were then isolated from the population of sorted cells. Clone AB1-L9 (5×10⁶) were inoculated in BALB/c mice in 100 μl of PBS. When tumor volume reached 200 mm³, tumors were excised. Digested tumors were prepared as previously described⁴³. To make single clones of AB1-L9 cell, digested tumors were diluted (0.5 cells/100 μl) and aliquoted 100 μl on 96 well-culture dish with selection reagent. Fifteen single clones were obtained, and clones with the highest GeoMean value were selected. The final clone was injected subcutaneously on BALB/c mice, and it was confirmed that more than 95% tumors were grown in BALB/c mice.

### B Cell ELISpot

Basement membrane (BM) was extracted from the femurs of eight immunized mice. BM was washed, filtered through a 70 mm mesh and lazed to eliminate RBC. Cells were resuspended in warm RPMI supplemented with heat inactivated FCS, 1% L-Glutamine and 1% Penicillin/Streptomycin. PVDF plates (0.45 um) (Mabtech) were coated with 2m/ml of LMB-100 for 18 hours, washed and blocked with assay media at 37° C. for 2 hours. Six replicas of each BM sample were seeded at a concentration of 100,000 cells/well and incubated for 4 hours. Spots that indicate anti-LMB-100 antibody secreting B cells were detected using a capture anti-mouse Ig biotinylated antibody (Mabtech) followed by ALP and BCIP/NTP substrate (KPL).

Spots were counted by computer-assisted image analysis (Immunospot5.0; Cellular Technology Limited). Results are shown in SFC/1E6 cells.

### Flow Cytometry

Spleens were dissected from mice immunized with either Alexa 488 labeled LMB-100, Cy5 labeled SVP-R or both or an untreated mouse. Splenocytes were extracted by injecting 3 ml of media supplemented with liberase (Roche), DNAas (Roche) and collagenase (Roche) to the spleen followed by 10 minutes incubation in 37° C. Spleens were minced, passed through a 70 mm mesh, washed and RBC were lysed. All cells were >90% viable by trypen blue. Cells were fixed, washed and stained as previously described⁴⁴ using the following antibodies obtained from Biolegend: CD3 (clone 17A2), CD4 (clone GK1.5), CD8 (clone 53-5.8), CD19 (clone 6D5), B220 (clone RA3, 6B2), CD11c (clone N418), IAIE (clone M5/114.15.2), CD11b (clone M1/70), Ly6G (clone 1A85), and Ly6C (clone HK1.4). Data was collected on a FACS CANTO II flow cytometer (BD Bioscience) and analyzed with FLOWJO version X (Treestar).

### Statistical Analysis

Statistical analysis and graphing were calculated using Graph Pad Prism. For multiple comparison of parametric variable, one-way analysis of variance (ANOVA) was used. For comparison of two non-parametric variables, Mann-Whitney was used and for multiple non-parametric variables, Friedman test with Dunn's multiple comparisons were used.

### Example 4: Rapamycin-Comprising Nanocarriers Prevent Long-Term LMB-100 Immunogenicity

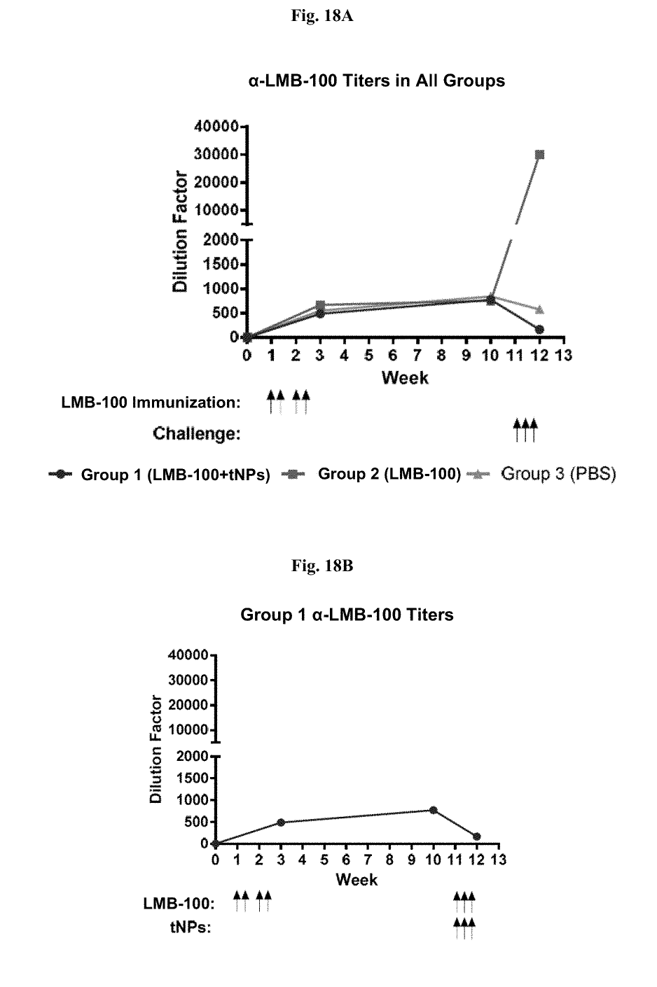

As shown in FIG. 17, administration of both LMB-100 and synthetic nanocarriers comprising rapamycin inhibited anti-LMB-100 antibody responses. Additionally, and importantly, synthetic nanocarriers comprising rapamycin did not enhance tumor growth as compared to PBS (FIG. 12F).

In order to evaluate the effectiveness of the LMB-100 and rapamycin-comprising nanocarrier combination in preventing long-term memory recall responses the time between the initial immune response and the LMB-100 and rapamycin-comprising nanocarrier challenge was increased. Female immune-competent BALB/c mice were treated according to the following schedule (Table 2):

There were 8 mice in each group. Doses were 50 μg/mL LMB-100 and 100 μL of rapamycin-comprising nanocarriers (intravenously, nanocarriers injected first.) Serum was isolated from blood samples and analyzed for anti-LMB-100 antibodies by ELISA. Sera from the second bleed were analyzed for anti-LMB-100 antibodies, and then mice were grouped such that each group had similar average anti-LMB-100 antibody titers before week 11 treatments.

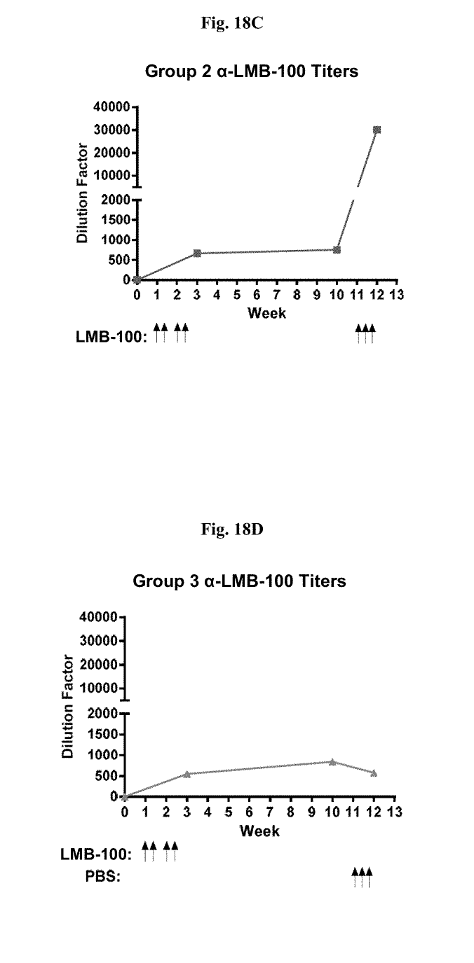

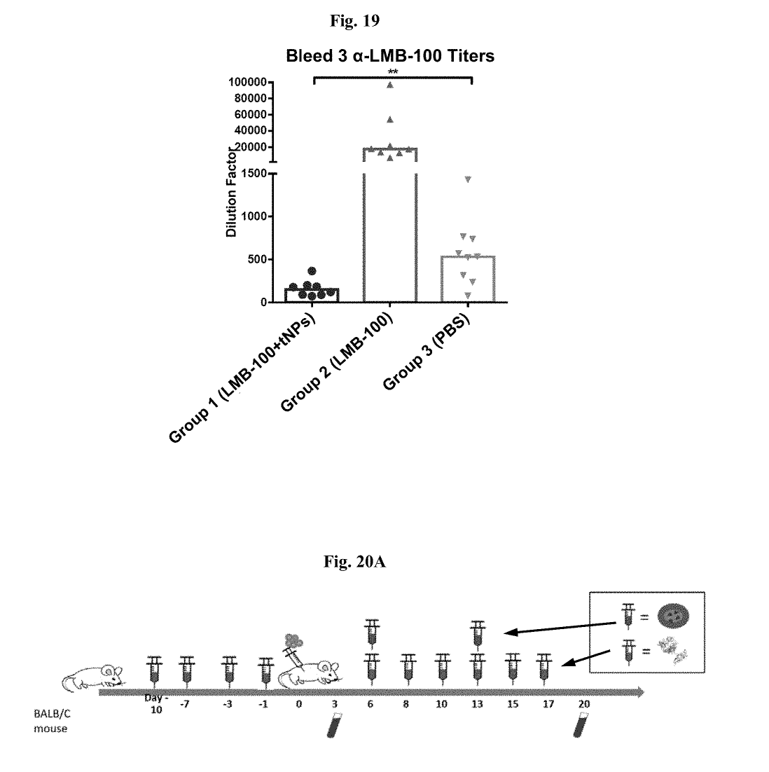

The serum samples from before and after challenge were analyzed; the results are shown in FIGS. 18A-18D and 19. The anti-LMB-100 antibody titers did not decline during the eight weeks following the primary immunization. In Group 1, challenge with LMB-100 and rapamycin-comprising nanocarriers significantly reduced the anti-LMB-100 antibody titer (bleed 3), compared to the pre-challenge titer (bleed 2) (Mann-Whitney test, p<0.005). The PBS challenge was found to have no effect on antibody titer (Mann-Whitney test, p>0.05). Further, challenge with LMB-100 and rapamycin-comprising nanocarriers significantly reduced the anti-LMB-100 antibody titer compared to the PBS-challenged and LMB-100-challenged controls (Mann-Whitney test, p<0.005).

### Example 5: Syngeneic Tumor Mouse Models

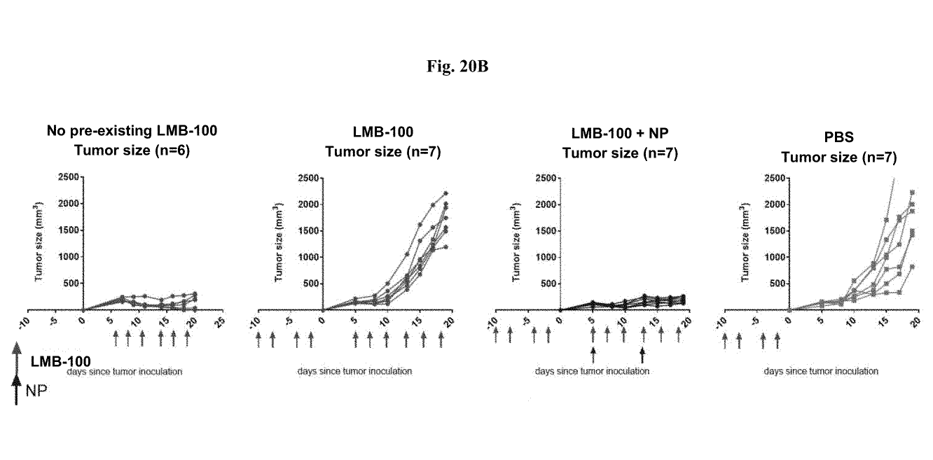

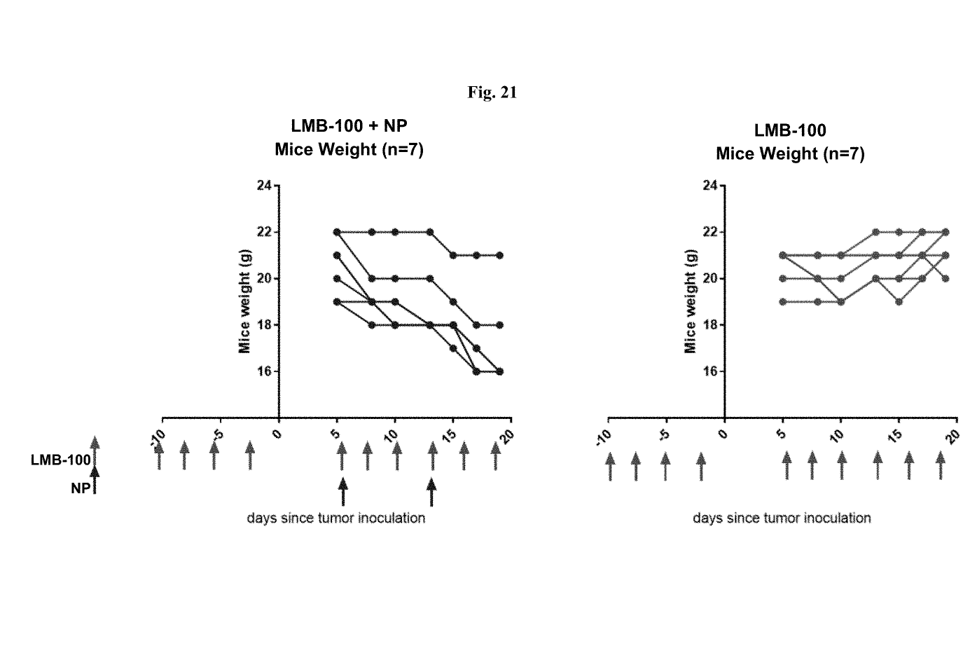

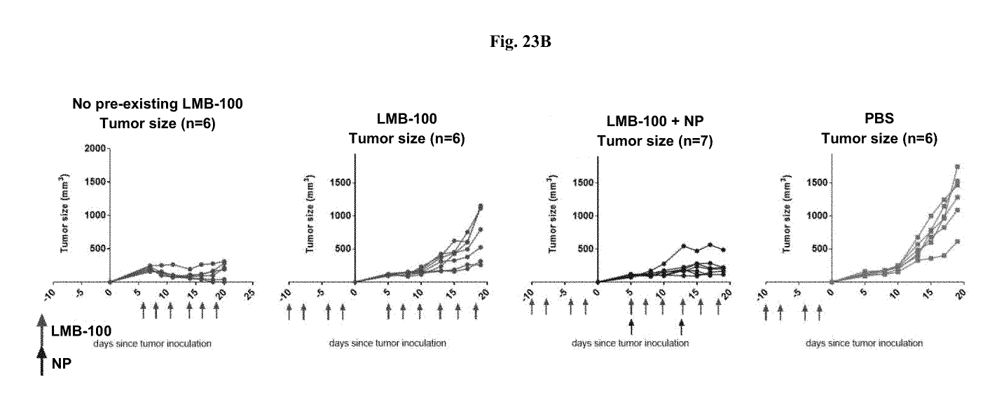

Two mouse models, BALB/c and a transgenic mouse that expresses human mesothelin in its genome and some cells, were immunized according to the schedules illustrated in FIGS. 20A and 23A. The pre-existing antibody syngeneic BALB/c mouse model was first investigated (FIG. 20A). The results (FIG. 20B) showed that LMB-100 had good anti-tumor activity on AB-1 cells, while pre-existing antibodies induced a dramatic neutralizing effect on LMB-100, resulting in a loss of efficacy. Administration of LMB-100 with rapamycin-comprising nanocarriers (1 mg/mL in an injection volume of 50 μL) prevented the formation of ADAs, resulting in a dramatic restoration of anti-tumor activity. However, this regimen resulted in weight loss in subjects (FIG. 21).

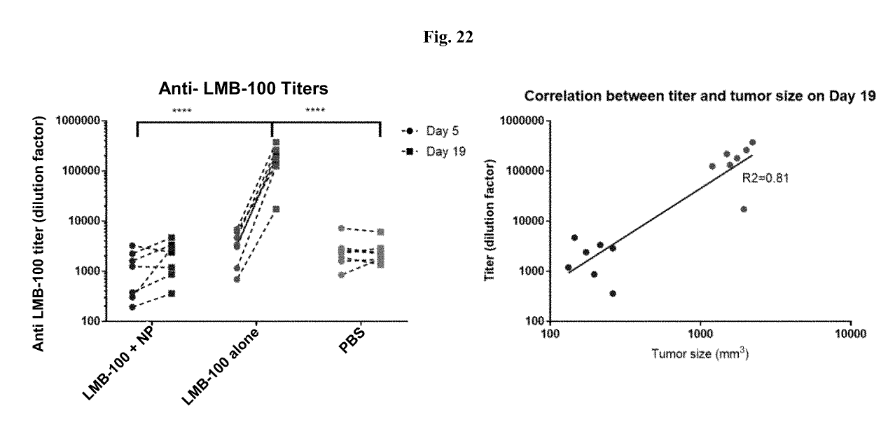

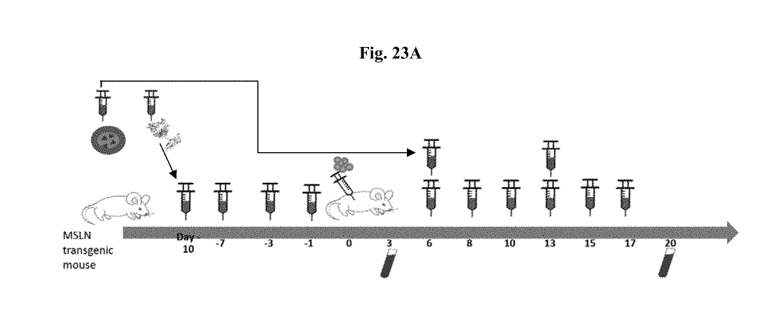

With respect to antibody titers (FIG. 22), all three groups started with similar average titers on day 5. After six doses of LMB-100, titers increased by 500-fold. Importantly, the combination of LMB-100 (six times) and rapamycin-comprising nanocarriers (two times) resulted in no significant change in titers, similar to the results seen in the vehicle control group. A correlation between titers and LMB-100 efficacy (as reflected by tumor size) was observed.

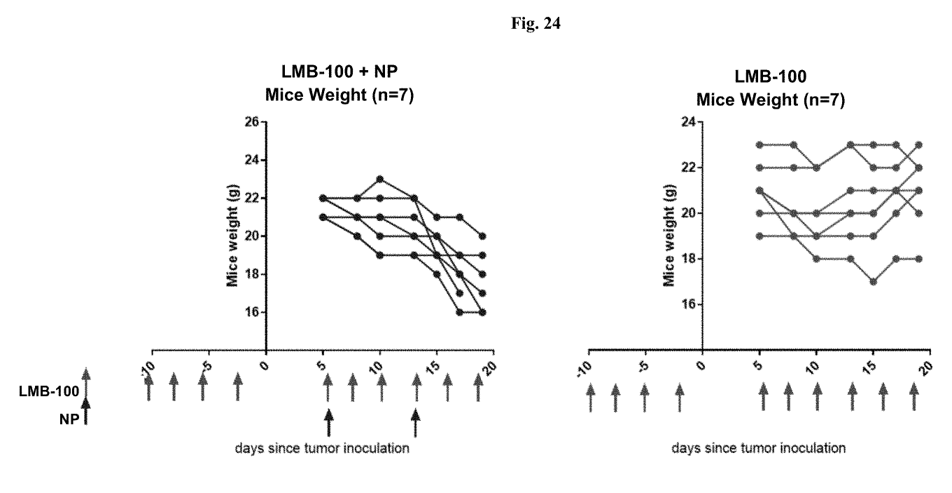

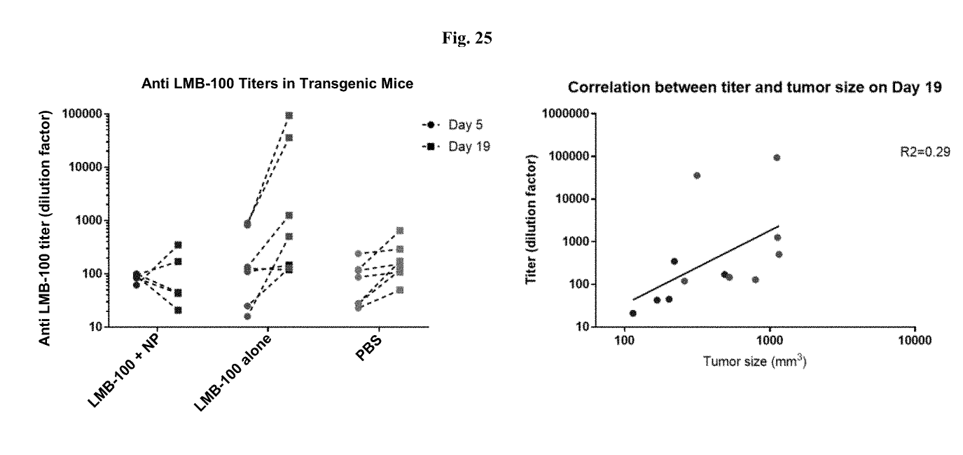

Using the transgenic mouse model, a similar protocol was followed (FIG. 23A). Similar results to those seen in the BALB/c model were noted (FIG. 23B). With respect to mouse weight, mice in the combination group were observed to lose weight (FIG. 24). The LMB-100 dose was lower (40 μg/mouse) in this model, and one of the seven mice treated with LMB-100 and rapamycin-comprising synthetic nanocarriers died on day 15. With respect to antibody titers, a difference between the titers of the different treatments groups was observed in the transgenic model (FIG. 25). However, the overall titers were lower in this model than in the BALB/c model.

### Example 6: Administration of Immunotoxin and Checkpoint Inhibitor

Mice were treated weekly with LMB-100 with or without synthetic nanocarriers comprising rapamycin on the first day of each week. Groups 2 and 3 also received anti-CLTA4 antibody on the fifth day of each week. The results show that mice receiving LMB-100 alone (Group 1) develop a titer of approximately 2000 at 5 weeks. Adding anti-CTLA4 to the LMB-100 regimen substantially increased the anti-LMB-100 response (Group 2). Surprisingly, administering LMB-100 with synthetic nanocarriers comprising rapamycin inhibited the anti-toxin antibody response even in the presence of an immunostimulating checkpoint inhibitor (Group 3) (FIG. 16A). Therefore, the synthetic nanocarriers comprising rapamycin are not adversely affected by an immunostimulatory checkpoint inhibitor in these subjects.

### REFERENCES

1. Maus, M. V. et al. T cells expressing chimeric antigen receptors can cause anaphylaxis in humans. Cancer Immunol. Res. 1, 26-31 (2013).
2. Avramis, V. I. & Tiwari, P. N. Asparaginase (native ASNase or pegylated ASNase) in the treatment of acute lymphoblastic leukemia. Int. J. Nanomedicine 1, 241-254 (2006).
3. Chung, C. H. et al. Cetuximab-induced anaphylaxis and IgE specific for galactose-alpha-1,3-galactose. N. Engl. J. Med. 358, 1109-1117 (2008).
4. Kreitman, R. J., Hassan, R., Fitzgerald, D. J. & Pastan, I. Phase I trial of continuous infusion anti-mesothelin recombinant immunotoxin SS1P. Clin. Cancer Res. 15, 5274-5279 (2009).
5. Sun, J. Y., Chatterjee, S. & Wong, K. K., Jr. Immunogenic issues concerning recombinant adeno-associated virus vectors for gene therapy. Curr. Gene Ther. 2, 485-500 (2002).
6. Baker, M. P., Reynolds, H. M., Lumicisi, B. & Bryson, C. J. Immunogenicity of protein therapeutics: The key causes, consequences and challenges. Self Nonself 1, 314-322 (2010).
7. Hassan, R. et al. Phase 1 study of the antimesothelin immunotoxin SS1P in combination with pemetrexed and cisplatin for front-line therapy of pleural mesothelioma and correlation of tumor response with serum mesothelin, megakaryocyte potentiating factor, and cancer antigen 125. Cancer 120, 3311-3319 (2014).
8. Kreitman, R. J. Recombinant immunotoxins for the treatment of chemoresistant hematologic malignancies. Curr. Pharm. Des. 15, 2652-2664 (2009).
9. Pastan, I., Hassan, R., Fitzgerald, D. J. & Kreitman, R. J. Immunotoxin therapy of cancer. Nat. Rev. Cancer 6, 559-565 (2006).
10. Kreitman, R. J. et al. Phase I trial of anti-CD22 recombinant immunotoxin moxetumomab pasudotox (CAT-8015 or HA22) in patients with hairy cell leukemia. J. Clin. Oncol. 30, 1822-1828 (2012).
11. Kreitman, R. J. et al. Phase I trial of recombinant immunotoxin anti-Tac(Fv)-PE38 (LMB-2) in patients with hematologic malignancies. J. Clin. Oncol. 18, 1622-1636 (2000).
12. Mazor, R., Onda, M. & Pastan, I. Immunogenicity of therapeutic recombinant immunotoxins. Immunol. Rev. 270, 152-164 (2016).
13. Hassan, R. et al. Phase I study of SS1P, a recombinant anti-mesothelin immunotoxin given as a bolus I.V. infusion to patients with mesothelin-expressing mesothelioma, ovarian, and pancreatic cancers. Clin. Cancer Res. 13, 5144-5149 (2007).
14. Hassan, R. et al. Major cancer regressions in mesothelioma after treatment with an antimesothelin immunotoxin and immune suppression. Sci. Transl. Med. 5, 208ra147 (2013).
15. Liu, W. et al. Recombinant immunotoxin engineered for low immunogenicity and antigenicity by identifying and silencing human B-cell epitopes. Proc. Natl. Acad. Sci. USA 109, 11782-11787 (2012).
16. Alewine, C. et al. Efficacy of RG7787, a next-generation mesothelin-targeted immunotoxin, against triple-negative breast and gastric cancers. Mol. Cancer Ther. 13, 2653-2661 (2014).
17. Stallone, G. et al. mTOR inhibitors effects on regulatory T cells and on dendritic cells. J. Transl. Med. 14, 152 (2016).
18. Mossoba, M. E. et al. Pentostatin plus cyclophosphamide safely and effectively prevents immunotoxin immunogenicity in murine hosts. Clin. Cancer Res. 17, 3697-3705 (2011).
19. Kishimoto, T. K. et al. Improving the efficacy and safety of biologic drugs with tolerogenic nanoparticles. Nat. Nanotechnol. 11, 890-899 (2016).
20. Zhang, A. H., Rossi, R. J., Yoon, J., Wang, H. & Scott, D. W. Tolerogenic nanoparticles to induce immunologic tolerance: prevention and reversal of FVIII inhibitor formation. Cell Immunol. 301, 74-81 (2016).
21. Maldonado, R. A. et al. Polymeric synthetic nanoparticles for the induction of antigenspecific immunological tolerance. Proc. Natl. Acad. Sci. USA 112, E156-165 (2015).
22. Getts, D. R., Shea, L. D., Miller, S. D. & King, N. J. Harnessing nanoparticles for immune modulation. Trends Immunol. 36, 419-427 (2015).
23. Setiady, Y. Y., Coccia, J. A. & Park, P. U. In vivo depletion of CD4+FOXP3+ Treg cells by the PC61 anti-CD25 monoclonal antibody is mediated by FcgammaRIII+ phagocytes. Eur. J. Immunol. 40, 780-786 (2010).
24. Onda, M. et al. Tofacitinib suppresses antibody responses to protein therapeutics in murine hosts. J. Immunol. 193, 48-55 (2014).
25. Youn, J. I., Nagaraj, S., Collazo, M. & Gabrilovich, D. I. Subsets of myeloid-derived suppressor cells in tumor-bearing mice. J. Immunol. 181, 5791-5802 (2008).
26. Youn, J. I. & Gabrilovich, D. I. The biology of myeloid-derived suppressor cells: the blessing and the curse of morphological and functional heterogeneity. Eur. J. Immunol. 40, 2969-2975 (2010).
27. Mezzapelle, R. et al. Human malignant mesothelioma is recapitulated in immunocompetent BALB/c mice injected with murine AB cells. Sci. Rep. 6, 22850 (2016).
28. Hassan, R., Williams-Gould, J., Watson, T., Pai-Scherf, L. & Pastan, I. Pretreatment with rituximab does not inhibit the human immune response against the immunogenic protein LMB-1. Clin. Cancer Res. 10, 16-18 (2004).
29. Maldonado, R. A. & von Andrian, U. H. How tolerogenic dendritic cells induce regulatory T cells. Adv. Immunol. 108, 111-165 (2010).
30. Kreitman, R. J. et al. Phase I trial of recombinant immunotoxin RFB4(dsFv)-PE38 (BL22) in patients with B-cell malignancies. J. Clin. Oncol. 23, 6719-6729 (2005).
31. Velazquez, V. M. et al. Effective depletion of pre-existing Anti-AAV antibodies requires broad immune targeting. Mol. Ther. Methods Clin. Dev. 4, 159-168 (2017).
32. Law, B. K. Rapamycin: an anti-cancer immunosuppressant? Crit. Rev. Oncol. Hematol. 56, 47-60 (2005).
33. O'Donnell, J. S., Massi, D., Teng, M. W. L. & Mandala, M. PI3K-AKT-mTOR inhibition in cancer immunotherapy, redux. Semin. Cancer Biol. doi: 10.1016/j.semcancer.2017.04.015.
34. Antignani, A. et al. Chemical screens identify drugs that enhance or mitigate cellular responses to antibody-toxin fusion proteins. PLoS One 11, e0161415 (2016).
35. Maeda, H. Tumor-selective delivery of macromolecular drugs via the EPR effect: background and future prospects. Bioconjug. Chem. 21, 797-802 (2010).
36. Dreaden, E. C., Austin, L. A., Mackey, M. A. & El-Sayed, M. A. Size matters: gold nanoparticles in targeted cancer drug delivery. Ther. Deliv. 3, 457-478 (2012).
37. Leshem, Y. et al. Combining local immunotoxins targeting mesothelin with CTLA-4 blockade synergistically eradicates murine cancer by promoting anti-cancer immunity. Cancer Immunol. Res. 5, 685-694 (2017).
38. Selby, M. J. et al. Anti-CTLA-4 antibodies of IgG2a isotype enhance antitumor activity through reduction of intratumoral regulatory T cells. Cancer Immunol. Res. 1, 32-42 (2013).
39. Piconese, S., Valzasina, B. & Colombo, M. P. OX40 triggering blocks suppression by regulatory T cells and facilitates tumor rejection. J. Exp. Med. 205, 825-839 (2008).
40. Guo, Z. et al. PD-1 blockade and OX40 triggering synergistically protects against tumor growth in a murine model of ovarian cancer. PLoS One 9, e89350 (2014).
41. Bauss, F. et al. Characterization of a re-engineered, mesothelin-targeted Pseudomonas exotoxin fusion protein for lung cancer therapy. Mol. Oncol. 10, 1317-1329 (2016).
42. Mazor, R. et al. Elimination of murine and human T-cell epitopes in recombinant immunotoxin eliminates neutralizing and anti-drug antibodies in vivo. Cell. Mol. Immunol. 14, 432-442 (2017).
43. Hollevoet, K. et al. In vitro and in vivo activity of the low-immunogenic antimesothelin immunotoxin RG7787 in pancreatic cancer. Mol. Cancer Ther. 13, 2040-2049 (2014).
44. Cuburu, N. et al. Intravaginal immunization with HPV vectors induces tissue-resident CD8+ T cell responses. J. Clin. Invest. 122, 4606-4620 (2012).

1. A method for treating a subject with a cancer, comprising:
a) creating a neoplasia-neutral tolerogenic environment in the subject, and
b) administering recombinant immunotoxin to the subject to treat the cancer.

2. The method of claim 1, wherein the cancer is a non-hematologic cancer.

3-4. (canceled)

5. The method of claim 1, wherein the recombinant immunotoxin when administered to the subject, or a test subject, without any immunosuppressive therapy generates or is expected to generate an unwanted immune response in the subject, or test subject.

6. The method of claim 1, wherein the recombinant immunotoxin when administered to the subject, or a test subject, without any synthetic nanocarriers comprising an immunosuppressant generates or is expected to generate an unwanted immune response in the subject, or test subject.

7. (canceled)

8. The method of claim 1, wherein the neoplasia-neutral tolerogenic environment in the subject is created by administration of synthetic nanocarriers comprising an immunosuppressant to the subject.

9. (canceled)

10. The method of claim 1, wherein the administration of the recombinant immunotoxin is repeated.

11-19. (canceled)

20. The method of claim 8, wherein the administration(s) of the synthetic nanocarriers comprising an immunosuppressant are concomitant with an administration of the recombinant immunotoxin.

21-22. (canceled)

23. The method of claim 8, wherein the method further comprises administering the recombinant immunotoxin without the synthetic nanocarriers comprising an immunosuppressant.

24-27. (canceled)

28. The method of claim 1, wherein the recombinant immunotoxin comprises an antibody, or antigen-binding fragment thereof, and a toxin.

29. The method of claim 1, wherein the ligand of the recombinant immunotoxin specifically binds an antigen expressed on cells of the cancer.

30-34. (canceled)

35. The method of claim 1, wherein the method further comprises administering a checkpoint inhibitor concomitantly with at least one administration of the recombinant immunotoxin.

36-49. (canceled)

50. The method of claim 6, wherein the immunosuppressant is an mTOR inhibitor.

51. (canceled)

52. The method of claim 6, wherein the immunosuppressant is encapsulated in the synthetic nanocarriers.

53. The method of claim 6, wherein the synthetic nanocarriers comprise polymeric nanocarriers.

54-57. (canceled)

58. The method of claim 6, wherein the mean of a particle size distribution obtained using dynamic light scattering of a population of the synthetic nanocarriers is a diameter greater than 110 nm.

59-61. (canceled)

62. The method of claim 58, wherein the diameter is less than 5 μm.

63-71. (canceled)

72. The method of claim 6, wherein the load of immunosuppressant comprised in the synthetic nanocarriers, on average across the synthetic nanocarriers, is between 0.1% and 50% (weight/weight).

73-76. (canceled)

77. The method of claim 6, wherein an aspect ratio of a population of the synthetic nanocarriers is greater than 1:1, 1:1.2, 1:1.5, 1:2, 1:3, 1:5, 1:7 or 1:10.

78. (canceled)

79. The method of claim 1, wherein the administering is by intravenous, intraperitoneal, or subcutaneous administration.

80. A kit comprising:
one or more doses comprising a recombinant immunotoxin and one or more doses comprising synthetic nanocarriers comprising an immunosuppressant.

81-85. (canceled)
